SEQUENCE LISTING (8 sequences)

SEQ ID NO: 1 - DNA, 25 bp, Artificial Sequence
  source 1..25: /mol_type=“DNA” /note=“forward primer for KIR3DL2 mRNA” /organism=“Artificial Sequence”
caacttctccatcggtcccttgatg

SEQ ID NO: 2 - DNA, 21 bp, Artificial Sequence
  source 1..21: /mol_type=“DNA” /note=“reverse primer for KIR3DL2 mRNA” /organism=“Artificial Sequence”
gtttgaccacacgcagggcag

SEQ ID NO: 3 - DNA, 21 bp, Artificial Sequence
  source 1..21: /mol_type=“DNA” /note=“forward primer for KIR3DL1 mRNA” /organism=“Artificial Sequence”
ggacatcgtggtcacaggtcc

SEQ ID NO: 4 - DNA, 25 bp, Artificial Sequence
  source 1..25: /mol_type=“DNA” /note=“reverse primer for KIR3DL1 mRNA” /organism=“Artificial Sequence”
gcctggaatgttctgttgaccttgc

SEQ ID NO: 5 - DNA, 21 bp, Artificial Sequence
  source 1..21: /mol_type=“DNA” /note=“CpG class A or ODN 2336; nucleotides from the sequence gac gacgtcgtg are phosphodiester nucleotides, the other nucleotides a re phosphorothioate nucleotides” /organism=“Artificial Sequence”
ggggacgacgtcgtggggggg

SEQ ID NO: 6 - DNA, 24 bp, Artificial Sequence
  source 1..24: /mol_type=“DNA” /note=“CpG class B or ODN 2006; all nucleotides are phosphorothio ate nucleotides” /organism=“Artificial Sequence”
tcgtcgttttgtcgttttgtcgtt

SEQ ID NO: 7 - DNA, 22 bp, Artificial Sequence
  source 1..22: /mol_type=“DNA” /note=“CpG class C or ODN 2395; all nucleotides are phosphorothio ate nucleotides” /organism=“Artificial Sequence”
tcgtcgttttcggcgcgcgccg

SEQ ID NO: 8 - DNA, 22 bp, Artificial Sequence
  source 1..22: /mol_type=“DNA” /note=“GpC or ODN 2395 control; all nucleotides are phosphorothio ate nucleotides” /organism=“Artificial Sequence”
tgctgcttttggggggcccccc

(19) United States
(12) Patent Application Publication
(10) Pub. No.: US 2018/0291102 A1
(43) Pub. Date: Oct. 11, 2018

(54) KIR3DL2 IS A BIOMARKER AND A THERAPEUTIC TARGET USEFUL FOR RESPECTIVELY PREVENTING AND TREATING A SUBSET OF CUTANEOUS AND NON-CUTANEOUS PERIPHERAL T-CELL LYMPHOMAS

(71) Applicants: INSTITUT NATIONAL DE LA SANTE ET DE LA RECHERCHE MEDICALE, Paris (FR); ASSISTANCE PUBLIQUE - HOPITAUX DE PARIS, Paris (FR); UNIVERSITE PARIS-EST CRETEIL VAL DE MARNE, Creteil (FR); UNIVERSITE PARIS DIDEROT - PARIS 7, Paris (FR)

(72) Inventors: Philippe GAULARD, Creteil (FR); Nicolas ORTONNE, Creteil (FR); Anne MARIE-CARDINE, Paris (FR); Armand BENSUSSAN, Paris (FR)

(21) Appl. No.: 15/987,125

(22) Filed: May 23, 2018

Related U.S. Application Data
(63) Continuation of application No. 14/892,779, filed on Nov. 20, 2015.

(30) Foreign Application Priority Data
May 29, 2013 (EP) PCT/EP2013/061107

(51) Int. Cl. C07K 16/28; G01N 33/574; C07K 16/30; C12N 15/117

(52) U.S. Cl. CPC C07K 16/2803; G01N 2500/10; G01N 33/57492; C07K 16/3061; C12N 15/117; C12N 2310/17; C12N 2320/30; G01N 33/57426; C07K 2317/73

(57) ABSTRACT

The present invention relates to a ligand molecule that specifically binds to KTR3DL2 at the surface of KTR3DL2 expressing malignant T-cells for the treatment of lymphomas. It also relates to the in vitro use of a level of expression of KIR3DL2 is a biomarker useful for diagnosing and/or monitoring a lymphoma.

### FIELD OF THE INVENTION

The present invention relates to the field of diagnosis or therapeutic treatment of T-cell lymphomas.

### BACKGROUND OF THE INVENTION

Peripheral T-cell lymphomas (PTCLs) are heterogeneous and uncommon non-Hodgkin's tumor diseases characterized by an aggressive clinical course and a mostly poor outcome with current treatment strategies.

One of the most urgent difficulties is to properly classify these diseases in order to provide the patient with the most adapted treatment, such as cell-targeted chemotherapy, radiotherapy and/or marrow bone transplantation.

Several attempts for classifying the PTCLs diseases have been made in the past, taking into account clinical symptoms and gene expression profiles.

Today, the World Health Organization recognizes several subtypes of PTCLs, as registered at the International Classification of Diseases for Oncology (ICDO, 3^{rd} Edition), such as (i) disseminated lymphomas; (ii) cutaneous lymphomas; (iii) nodal non-cutaneous lymphomas; and (iv) extra nodal non-cutaneous lymphomas.

Among the cutaneous PTCL, Sézary syndrome and transformed mycosis fungoides are the most prevalent cutaneous T-cell lymphomas. Sézary syndrome presents an aggressive clinical behaviour, with widespread skin involvement, resulting in an erythroderma, enlarged lymph nodes and the presence of a significant number of malignant lymphocytes, called Sézary cells. Oppositely, mycosis fungoides presents an indolent clinical behaviour but in about 10% of patients, the disease progresses to a large T-cell lymphoma (“transformed mycosis fungoides”) resulting in large skin, often ulcerated, skin tumours, sometimes with lymph nodes or internal organs involvement.

Although there is currently no cure for Sézary syndrome and transformed mycosis fungoides, several palliative approaches can be undertaken in order to ameliorate the patient's life. For example, these approaches comprise:
drug therapy and chemotherapy, including topical corticosteroids, imiquimod, retinoids bexarotene, interferon-alpha, histone deacetylase inhibitors (HDACi, such as vorinostat and romidepsin), oral methotrexate, denileukin diftitox (an antineoplastic agent, combining Interleukin-2 and diphtheria toxin), proteasome inhibitors, immunomodulatory agents (lenalidomide);
phototherapy, including UVB phototherapy;
photodynamic therapy, including psoralen+ultraviolet A (PUVA);
radiotherapy;
total skin electron beam (TSEB);
extracorporeal photopheresis (ECP);
autologous stem cell transplantation;
allogenic stem cell transplantation.

Most of these approaches present limitations mostly because of the side effects that are endured by normal non-pathogenic cells and may affect the patient's benefits of such treatments.

There is hence a need for targeted therapy. This concept has been recently implemented by using alemtuzumab, a monoclonal antibody directed towards CD52, a peptide found at the surface of mature lymphocytes, monocytes and dendritic cells (Lundin et al. Phase 2 study of alemtuzumab (anti-CD52 monoclonal antibody) in patients with advanced mycosis fungoides/Sezary syndrome. Blood. 2003 Jun 1; 101(11):4267-72).

Previous studies demonstrated that treatment of Sézary cells with the Janus kinase (JAK) inhibitor tyrphostin AG490, as well as with Cucurbitacin I or Curcumin, efficiently promotes phospho-STAT3 dephosphorylation and induces Sézary cell apoptosis (Eriksen et al. Constitutive STAT3-activation in Sezary syndrome: tyrphostin AG490 inhibits STAT3-activation, interleukin-2 receptor expression and growth of leukemic Sezary cells. Leukemia. 2001; 15:787-793; van Kester et al. Cucurbitacin I inhibits Stat3 and induces apoptosis in Sezary cells. J Invest Dermatol. 2008; 128:1691-1695; Zhang et al. Curcumin selectively induces apoptosis in cutaneous T-cell lymphoma cell lines and patients' PBMCs: potential role for STAT-3 and NF-kappaB signaling. J Invest Dermatol. 2010;130:2110-2119).

Regarding some physiological aspects of Sezary's syndrome, it has notably been shown in the art that, among the biomarkers that may be relevant in Sezary's individuals, a combination of 4 biomarkers are of special interest, since PSL3, TWIST, KIR3DL2 and NKp46 were shown to be overexpressed in T-cells from Sézary and transformed mycosis fungoides individuals (Michel et al. Combination of PSL3, Twist, CD158/KIR3DL2 and NKp46 gene expression for the diagnosis of Sezary syndrome. J Invest Dermatol. 2012; 132(2), page S50).

However, there are confusing data whenever KIR3DL2 should be considered as a relevant biomarker for Sézary syndrome and transformed mycosis fungoides.

Indeed, Bagot et al. (CD4(+) cutaneous T-cell lymphoma cells express the p140-killer cell immunoglobulin-like receptor. Blood. 2001 Mar 1; 97(5):1388-91), Poszepczynska-Guigné et al. (CD158k/KIR3DL2 is a new phenotypic marker of Sezary cells: relevance for the diagnosis and follow-up of Sezary syndrome. J Invest Dermatol. 2004 Mar; 122(3):820-3) and Ortonne et al. (CD158k/KIR3DL2 and NKp46 are frequently expressed in transformed mycosis fungoides. Exp Dermatol. 2012 Jun; 21(6):461-3) reported an overexpression of KIR3DL2 at the surface of T-cells from patients having a Sézary syndrome or a transformed mycosis fungoides.

In complete contradiction with the above studies, the expression of KIR3DL2 was also found in the art to be down-regulated in Sézary cells, when assessed by measuring the level of KIR3DL2 mRNA by quantitative RT-PCR (see WO 2007/071829).

Booken et al. (Sézary syndrome is a unique cutaneous T-cell lymphoma as identified by an expanded gene signature including diagnostic marker molecules CDO1 and DNM3. Leukemia. 2008 (22), 393-399) reported that KIR3DL2 is only expressed in a subpopulation of patient having a Sézary syndrome.

In addition, Iqbal et al. (Molecular signature to improve diagnosis in peripheral T-cell lymphoma and prognostication in angioimmunoblastic T-cell lymphoma; Blood, 2010, 115(5):1026-36) reported KIR3DL2 as a putative biomarker for a subset of PTCL “not otherwise specified” (PTCL/NOS), but remained silent about the cutaneous lymphoma, such as Sézary syndrome and transformed mycosis fungoides.

Finally, Nebozhyn et al. (Quantitative PCR on 5 genes reliably identifies CTCL patients with 5% to 99% circulating tumor cells with 90% accuracy. Blood. 2006 Apr 15;107(8):3189-96) suggested a set of 5 reliable biomarkers, i.e. STAT4, GATA-3, PLS3, CD1D, and TRAIL, hence discarding KIR3DL2 from being a valuable biomarker for the diagnosis of Sézary syndrome.

It is to be noted that KIR3DL2 was also found to be overexpressed at the surface of some T-cells from patient having been diagnosed with adult T-cell leukaemia (ATCL), as disclosed in Obama et al. (Killer cell immunoglobulin-like receptor/3DL2 expression in adult T-cell leukaemia. Br J Haematol. 2007 Sep; 138(5):666-7). However, according to this study, KIR3DL2 is believed not to be a highly specific biomarker for ATCL.

Killer immunoglobulin-like receptors (KIR) represent a family of receptors that are used by human Natural Killer (NK) cells and T-lymphocyte subsets to specifically recognize MHC class I molecules.

KIR3DL2 belongs to the KIR receptor family displaying 3 immunoglobulin-like domains and a long cytoplasmic tail.

KIR3DL2 has been reported to be a candidate for target therapy, since a monoclonal antibody that binds to KIR3DL2 is able to induce an antibody-dependent cellular cytotoxicity (ADCC) against malignant T-cells expressing KIR3DL2 (WO 2010/081890).

Furthermore, Sivori et al. (A novel KIR-associated function: evidence that CpG DNA uptake and shuttling to early endosomes is mediated by KIR3DL2; Blood, 2010, 116(10):1637-1647) discloses the use of CpG oligodeoxynucleoside to treat NK-cells (Natural killer) and to induce the NK-cells to produce and to release various cytokins.

There is a need in the art for novel therapeutic strategies against T-cell lymphomas, as well as for reliable tools for diagnosing these diseases.

### SUMMARY OF THE INVENTION

In a first aspect, the invention describes a ligand molecule, that specifically binds to KIR3DL2 for the prevention and/or the treatment of a KIR3DL2 expressing malignant T-cells lymphoma selected from the group comprising Sézary syndrome, transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

In one aspect, the invention relates to a ligand molecule, that specifically binds to KIR3DL2 for the prevention and/or the treatment of a KIR3DL2 expressing malignant T-cells lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In another aspect, the present invention describes a pharmaceutical composition comprising a ligand molecule as defined in the present invention and a pharmaceutically acceptable carrier for the prevention and/or the treatment of a KIR3DL2 expressing malignant T-cells lymphoma selected from the group comprising Sézary syndrome, transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

In one aspect, the present invention relates to a pharmaceutical composition comprising a ligand molecule as defined in the present invention and a pharmaceutically acceptable carrier for the prevention and/or the treatment of a KIR3DL2 expressing malignant T-cells lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

Another aspect of the invention describes a method for a treating KIR3DL2 expressing malignant T-cells lymphoma in an individual in need thereof, comprising administering to said individual a ligand molecule that specifically binds to KIR3DL2, wherein said lymphoma is selected from the group comprising transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

Another aspect of the invention retates to a method for a treating KIR3DL2 expressing malignant T-cells lymphoma in an individual in need thereof, comprising administering to said individual a ligand molecule that specifically binds to KIR3DL2, wherein said lymphoma is selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In another aspect of the present invention relates to a method for treating a KIR3DL2 expressing malignant T-cells lymphoma in an individual in need thereof, comprising administering to said individual a pharmaceutical composition comprising a ligand molecule that specifically binds to KIR3DL2, and a pharmaceutically acceptable carrier, wherein said lymphoma is selected from the group comprising transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma, preferably sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

A still further aspect of the invention relates to an in vitro use of a level of expression of KIR3DL2 as a biomarker for diagnosing and/or monitoring a lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In another aspect, the invention relates to an in vitro method for diagnosing and/or monitoring a lymphoma in an individual comprising at least a step of quantifying the level of expression of KIR3DL2, said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In another aspect, the invention also relates to a method for monitoring the effectiveness of treatment against a lymphoma, in an individual in need thereof, with a therapeutic agent, said method comprising the steps of:
(i) providing a pre-administration biological sample from an individual prior to administration of the therapeutic agent;
(ii) measuring the level of expression of KIR3DL2 in the pre-administration biological sample;
(iii) providing one or more post-administration biological samples from the individual;
(iv) measuring the level of expression of KIR3DL2 in the post-administration biological samples;
(v) comparing the level of expression of KIR3DL2 measured for the pre-administration biological sample with the level of expression of KIR3DL2 measured for the post-administration biological sample or samples; and
(vi) altering the administration of the therapeutic agent to the individual accordingly, wherein said lymphoma is selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In a further aspect, the present invention relates to a method for monitoring the effectiveness of treatment against a lymphoma, in an individual in need thereof, with a therapeutic agent, said method comprising the steps of:
(i) providing a pre-administration biological sample from an individual prior to administration of the therapeutic agent;
(ii) measuring the ratio of the levels of expression of KIR3DL2/KIR3DL1 in the pre-administration biological sample;
(iii) providing one or more post-administration biological samples from the individual;
(iv) measuring the ratio of the levels of expression of KIR3DL2/KIR3DL1 in the post-administration biological samples;
(v) comparing the ratio of the levels of expression of KIR3DL2/KIR3DL1 in the pre-administration biological sample with the ratio of the levels of expression of KIR3DL2/KIR3DL1 in the post-administration biological sample or samples; and
(vi) altering the administration of the therapeutic agent to the individual accordingly, wherein said lymphoma is selected from the group comprising sub-cutaneous panniculitis-like T-cell lymphoma, primary cutaneous CD8+ aggressive epidermotropic cytotoxic T-cell lymphoma, enteropathy-associated T-cell lymphoma, and hepatosplenic gamma-delta T-cell lymphoma.

In another aspect, the invention also relates to a method for adapting a treatment against a lymphoma in an individual in need thereof, wherein said method comprises at least the steps of:
a) performing, on at least one biological sample collected from said individual, the in vitro diagnosis method according to the present invention; and
b) adapting the treatment against the lymphoma of said individual by administering to said individual a suitable therapy,
wherein said lymphoma is selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

Another aspect of the invention relates to a method for screening a compound candidate that affects KIR3DL2 expression level, said method comprising the step of:
a) providing at least one T-cell able to express KIR3DL2;
b) measuring KIR3DL2 expression level by the at least one T-cell provided at step a), whereby a first KIR3DL2 expression value is obtained;
c) incubating the said KIR3DL2 expressing at least one T-cell with a candidate compound to be tested;
d) measuring the KIR3DL2 expression level by the KIR3DL2 expressing at least one T-cell of step c), whereby a second KIR3DL2 expression value is obtained;
e) comparing the said first KIR3DL2 expression value with the said second KIR3DL2 expression value; and
f) selecting the said candidate compound when the said second KIR3DL2 expression value is lower than the said first KIR3DL2 expression value.

A further aspect of the present invention relates to a method for the screening of a candidate compound that affects KIR3DL2 biological activity, said method comprising the step of:
a) providing at least one T-cell able to express KIR3DL2;
b) measuring KIR3DL2 biological activity in the at least one T-cell provided at step a), whereby a first activity value is obtained;
c) incubating KIR3DL2 expressing T-cell with a candidate compound to be tested;
d) measuring the KIR3DL2 biological activity in the KIR3DL2 expressing T-cell obtained at the end of step b), whereby a second activity value is obtained;
e) comparing the said first activity value with the said second activity value.

Finally, another aspect of the present invention relates to a kit for diagnosing and/or monitoring a lymphoma in an individual, which kit comprises means for quantifying the level of expression of KIR3DL2 or the levels of expression of KIR3DL1 and KIR3DL2, said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

The invention has for advantages to provide a simple, cost-effective, and reliable assay to diagnose and/or monitor a subset of cutaneous lymphomas and nodal and extra nodal non-cutaneous lymphomas.

According to another of its advantages, the invention allows for monitoring a therapeutic treatment presumed effective for preventing and/or treating a subset of cutaneous lymphomas and nodal and extra nodal non-cutaneous lymphomas or for screening drug candidate presumed effective for preventing and/or treating a subset of cutaneous lymphomas and nodal and extra nodal non-cutaneous lymphomas.

### LEGEND OF THE FIGURES

FIG. 1: KIR3DL2 protein expression in tissue samples of a subset of cutaneous PTCL.

The inventors assume that the labelling obtained with antibodies from the 5.133 clone (Miltenyi-Biotec), that specifically react with both KIR3DL2 and KIR3DL1, actually identified KIR3DL2 as RT-PCR studies showed no or a very low expression of KIR3DL1 transcripts, as compared to KIR3DL2.

(A) and (B). Sézary syndrome samples from the PHRC KIRs, with a dense neoplastic infiltrate (A) or a slight perivascular infiltrate (B) labelled with the anti-KIR3DL2 monoclonal antibody, showing both a membrane positivity with no background labelling. Epidermotropic neoplastic cells can be identified in A (arrowheads)

(C) and (D). A case of transformed mycosis fungoides (MF) showed strong membrane expression of KIR3DL2, including in epidermotropic neoplastic cells within the epidermis (Ep, arrowheads).

(E) and (F). Primary cutaneous CD8+ aggressive epidermotropic cytotoxic T-cell lymphoma (CTCL) showed diffuse expression of KIR3DL2 by the neoplastic cells, in both the dermis and the epidermotropic cells (arrowheads).

(G) and (H). Sub-cutaneous ‘panniculitis-like’ T-cell lymphoma (TCL) displayed scattered KIR3DL2+ cells. Some stained cells are in the vicinity of adipocytes where the neoplastic cells usually localize, but the staining is heterogeneous.

Original magnifications: A, B, D, F, G and H : ×200; C and E : ×100.

FIG. 2: KIR3DL2 expression in tissue samples of a subset of non-cutaneous PTCL. Although no data regarding KIR3DL1 transcripts are available in this group, the correlation between KIR3DL2 transcript detection using transcriptomic analyses and labelling with the 5.133 monoclonal antibody suggests that only KIR3DL2 was actually labelled at the surface of neoplastic T-cells.

(A) and (B). Peripheral T-cell lymphoma, not otherwise specified (PTCL/NOS) showed no significant expression of KIR3DL2, with only scattered positive cells, that may correspond to reactive CD8+ effector T-cells and/or natural killer cells (arrowheads).

(C) and (D). Enteropathy-associated T-cell lymphoma (EATL) showed a diffuse and strong KIR3DL2 positivity. An intra-tumoral vessel is seen (V), showing labelling neither in the endothelium (arrow) nor in the pericytes.

(E) and (F). Adult T-cell leukemia lymphoma (ATLL) showed a strong and diffuse expression of KIR3DL2. As in the preceding case, intra-tumoral vessels taken as negative controls are not stained (negative endothelial cells, arrow).

(G) and (H). Hepatosplenic T-cell lymphoma (HSTL) displayed a diffuse expression of KIR3DL2.

Original magnifications: A, B, E, G and H : ×200; D and F : ×100.

FIG. 3: KIR3DL2 transcript expression in hepatosplenic T-cell lymphomas (HSTL).

In this graph are presented the relative KIR3DL2 mRNA levels in the hepatosplenic T-cell lymphoma (HSTL), enteropathy-associated T-cell lymphoma (EATL) and adult T-cell leukemia lymphoma (ATLL) samples shown to be positive for KIR3DL1/2 using immunohistochemistry (grey), in all the HSTL and angioimmunoblastic T-cell lymphoma (AITL) samples from the PHRC TENOMIC group (white).

FIG. 4A-B: Down-modulation of KIR3DL2 expression on tumoral Sézary cells upon CpG ODN-C treatment.

(A) Peripheral blood mononuclear cells (PBMC) from Sézary patients were left untreated or incubated in the presence of CpG ODN-C (10 g/ml) or AZ158 mAb (2 μg/ml). After 24 h of incubation, cells were labelled with anti-KIR3DL2 mAb (Q66) plus FITC-conjugated goat anti-mouse IgM secondary antibodies, anti-TCRV-PE, -CD3-PC5 and CD4-PC7 mAbs. Shown are the TCRV β/KIR3DL2 stainings corresponding to the gated CD3+CD4+ T lymphocyte population. The mean fluorescence intensity (MFI) of KIR3DL2 labelling is indicated.

(B) Graphical representation of KIR3DL2 MFI observed on the CD3+CD4+ cells from Sézary patients (n=12) following incubation with CpG ODN-C, control (Ctrl) ODN or AZ158 mAb.

FIG. 5A-D: KIR3DL2 binding with AZ158 mAb, but not CpG ODN-C, down-modulates the CD3-induced proliferation and induces cell death of Sézary cells.

(A) PBMC from Sézary patient were pre-loaded with carboxyfluorescein succinimidyl ester (CFSE) and further left untreated or incubated with anti-CD3 mAb, AZ158 mAb or CpG ODN-C alone or in combination, as indicated. After 4 days of culture, cells were collected and subjected to flow cytometry analyses. Shown are the CFSE staining of the gated TCRVβ3+ CD4+ tumoral T cell clone.

(B) Cells treatment was conducted as in (A). Immunolabellings were performed using anti-TCRV3-PE and -CD4-FITC mAb and 7AAD. The % of early (7AADlow) and late (7AADhigh) apoptotic cells within the TCRV3+CD4+ tumoral cells are indicated. The results shown in (A) and (B) are representative of experiments performed on 4 Sézary patients.

(C) Freshly isolated Sézary syndrome cells were incubated with anti-CD3 and/or AZ158 mAb, as indicated. An isotype matched control mAb (anti-CD16) was used to equalize the amount of antibodies used in each condition. Cross-linking was induced by addition of goat anti-mouse Igs except for resting condition (NT). After lysis, the antibodies-targeted molecules were collected and the resulting immunoprecipitates subjected to electrophoresis and Western blotting procedures. The immunoblot was revealed with an anti- phospho-CD3 mAb (upper panel), and re-probed after de-hybridization using an anti-CD3 mAb to assess efficient immunoprecipitation (lower panel).

(D) Sézary patient CD4+ T-cells were activated with anti-CD3 alone or together with AZ158 mAb or CpG ODN-C and subjected to lysis. Post-nuclear lysates were prepared, resolved by SDS-10% PAGE and analyzed by Western blotting using an anti-phospho-Erk mAb. Equal loading of the samples was assessed after stripping and reprobing with anti-Erk1/2 antibodies.

FIG. 6A-C: CpG ODN-C induces malignant Sézary cells apoptosis.

(A) Sézary patient PBMCs were incubated with CpG ODN-C, AZ158 mAb or control ODN for 7 days at 37° C. Stainings for the detection of apoptotic TCRV β+ CD4+ tumoral T cells were performed as described in the legend of FIG. 5B.

(B) Graphical representation of the % of early (7AADlow) and late (7AADhigh) apoptotic cells within the TCRV+CD4+ population of Sézary patients (n=8) following incubation with CpG ODN-C or control ODN (Ctrl).

(C) Sezary cell line was incubated for the indicated time with CpG ODN-C. Post-nuclear lysates were prepared and processed for SDS-PAGE and immunoblotting. Blots were probed successively with anti-cleaved-caspase 7, -caspase 3, -PARP and Erk1/2 antibodies.

FIG. 7A-B: CpG ODN-C treatment of Sézary cells results in STAT3 dephosphorylation.

(A) Sézary cell line was incubated for the indicated time with CpG ODN-C. Cellular lysates were subjected to gel electrophoresis and transferred to nitrocellulose membrane. Blots were then revealed using anti-phospho-STAT3 antibodies, de-hybridized and re-probed with STAT3 antibodies.

(B) Sorted CD4+ T-cells (98% of which were KIR3DL2+) from a Sézary patient were incubated in medium alone or supplemented with CpG ODN-C, AZ158 mAb or control ODN for 24 h at 37° C. Lysates were then analysed as in (A).

FIG. 8A-C: GpC ODN treatment of Sézary cells results in apoptosis.

(A) PBMCs from a Sézary patient were incubated without any oligonucleotide (curve 1) or with CpG ODN-C (curve 2), GpC (curve 3) or control ODN (curve 4) for 7 days at 37° C. KIR3DL2 expression on the malignant CD4⁺ T cells (identified as TCR Vβ1+ cells) was assessed for each condition of incubation.

(B) PBMCs from a Sézary patient were incubated with CpG ODN-C, GpC or control ODN for 7 days at 37° C. Stainings for the detection of apoptotic TCRVβ1+ CD4+ tumoral T cells, i.e. early (7AADlow) and late (7AADhigh) apoptotic cells, were performed as previously described (see FIG. 5B).

(C) Percentages of early (7AADlow) and late (7AADhigh) apoptotic cells from (B) are plotted for each type of treatment.

### DETAILED DESCRIPTION OF THE INVENTION

Taking into account the high degree of discrepancy between the various experimental data available in the art, the present inventors have initiated experimental work ab initio with the view of determining the feasibility of providing diagnosis and/or therapeutic tools for T-cell lymphomas.

The present invention relies upon the findings that KIR3DL2 is found to be overexpressed at the surface of T-cells from several individuals having a cutaneous PTCL such as Sézary syndrome, transformed mycosis fungoides and adult T-cell leukaemia/lymphoma as well as, most surprisingly, a subset of both cutaneous and non-cutaneous nodal and extra nodal lymphomas.

To the inventor's knowledge, KIR3DL2 could be identified, for the first time, as a relevant and specific biomarker for two cutaneous PTCLs, such as sub-cutaneous panniculitis-like T-cell lymphoma and primary cutaneous CD8+ aggressive epidermotropic cytotoxic T-cell lymphoma, and two non-cutaneous PTCLs, namely enteropathy-associated T-cell lymphoma and hepatosplenic gamma-delta T-cell lymphoma.

However, KIR3DL2 could not be identified as a significant, relevant and specific biomarker for some cutaneous PTCLs, such as primary cutaneous CD30+ T-cell lymphoproliferative disorders, and for some non-cutaneous PTCLs, such as angioimmunoblastic T-cell lymphoma, anaplastic large cell lymphomas, both ALK negative and ALK positive, extranodal NK/T cell lymphomas nasal type and peripheral T-cell lymphomas not otherwise specified.Hence KIR3DL2 has revealed to consist of a relevant biomarker for diagnosing and/or monitoring these specific lymphomas, as well as an advantageous biomarker for providing a first screen of a defined subset of both cutaneous and non-cutaneous PTCL diseases.

KIR3DL2 As a Biomarker

Considering the results from Michel et al. (Combination of PSL3, Twist, CD158/KIR3DL2 and NKp46 gene expression for the diagnosis of Sezary syndrome. J Invest Dermatol. 2012; 132(2), page S50), Bagot et al. (CD4(+) cutaneous T-cell lymphoma cells express the p140-killer cell immunoglobulin-like receptor. Blood. 2001 Mar 1; 97(5):1388-91), Poszepczynska-Guigné et al. (CD158k/KIR3DL2 is a new phenotypic marker of Sezary cells: relevance for the diagnosis and follow-up of Sezary syndrome. J Invest Dermatol. 2004 Mar;122(3):820-3) Ortonne et al. (CD158k/KIR3DL2 and NKp46 are frequently expressed in transformed mycosis fungoides. Exp Dermatol. 2012 Jun; 21(6):461-3) and the teaching of WO 02/50122 and WO 2010/081890, in contradiction with the results from Booken et al. (Sézary syndrome is a unique cutaneous T-cell lymphoma as identified by an expanded gene signature including diagnostic marker molecules CDO1 and DNM3. Leukemia. 2008 (22), 393-399), Iqbal et al. (Molecular signature to improve diagnosis in peripheral T-cell lymphoma and prognostication in angioimmunoblastic T-cell lymphoma; Blood, 2010, 115(5):1026-36) and the teaching of WO 2007/071829, the inventors assessed the overexpression of KIR3DL2 in T-cells from Sézary syndrome and transformed mycosis fungoides.

As a negative control for KIR3DL2 expression, the inventors' first approach was to select other lymphoma types from the cutaneous PTCL sub-group, as well as lymphoma types from the non-cutaneous nodal and extra nodal PTCL sub-group. Because, at the time of the instant invention, systematic biomarker approaches performed by several research teams did not provide any specific correlation between a change in expression of KIR3DL2 and the occurrence of any of these PTCLs, except for Sézary syndrome, transformed mycosis fungoides and adult T-cell lymphoma, said other types of lymphomas were assumed not to express KIR3DL2 at the surface of the malignant T-cells.

As mentioned already, except for the fact that all PTCLs are characterized by alterations in the T-cell cellular type, their physiological, histological, clinical and prognosis features of are quite various and different, which explain the difficulty to classify these various diseases.

For example, cutaneous PTCLs, other than Sézary syndrome or transformed mycosis fungoides, which were discussed above, may be distinguished as follows:
primary cutaneous CD8+ aggressive epidermotropic cytotoxic T-cell lymphomas are aggressive T-cell type lymphomas featured by localized or disseminated eruptive papules, nodules or tumors that show central ulceration and necrosis or superficial, hyperkeratotic patches and plaques;
sub-cutaneous panniculitis-like T-cell lymphomas represent rare forms of indolent T-cell lymphomas; patients are often seen with solitary or multiple nodules and plaques, usually involving the legs; ulceration of the nodules and plaques are rather not common.

As for non-cutaneous PTCLs, one may distinguish:
adult T-cell leukaemias/lymphomas are associated with the human T-cell leukaemia virus-1, which may be transmitted during unprotected sexual activity, childbirth, breast feeding, blood transfusion; usually their prognosis is poor;
anaplastic large cell non cutaneous lymphomas are rare lymphomas, affecting mainly nodal sites; they are subdivided as ALK negative and ALK positive lymphomas, depending on, respectively, the absence or the presence of a protein called “anaplastic lymphoma kinase” (ALK); ALK negative patients usually require more aggressive treatment, whereas ALK positive patients are much responsive to chemotherapy;
angioimmunoblastic T-cell lymphomas are common PTCLs and present an aggressive course; they affect mainly lymph nodes, and may affect the skin, the liver or the spleen;
enteropathy-associated T-cell lymphomas are significantly associated with celiac disease, caused by a hypersensitivity to gluten; they are characterized by stomach pain, weight loss, gastrointestinal bleeding or bowel perforation;
extranodal NK/T cell lymphomas, nasal type, are affecting the nasal and the paranasal sinus areas behind the nose and the cheeks, and may affect also the skin, the gastrointestinal tract and testes; these lymphomas are associated with the Epstein-Barr virus;
hepatosplenic gamma-delta T-cell lymphomas are rare and aggressive diseases that originate from the liver or the spleen;
peripheral T-cell lymphomas, not otherwise specified are the most common PTCLs and comprise various aggressive T-cell lymphomas that cannot be encompassed by the other subcategories of PTCLs; most patients are affected at nodal sites, although extranodal sites may be also affected, such as the bone marrow, the liver, the gastrointestinal tract or the skin.

Frozen skin samples of patient with a cutaneous PTCL such as Sézary syndrome; transformed mycosis fungoides; primary cutaneous CD30+ T-cell lymphoproliferative disorder (cutaneous anaplastic large cell lymphoma and lymphomatoid papulosis); sub-cutaneous panniculitis-like T-cell lymphoma; primary cutaneous nasal-type NK/T-cell lymphoma; and primary cutaneous CD8+ aggressive epidermotropic cytotoxic T-cell lymphoma were assessed for KIR3DL2 overexpression at the surface of T-cells.

Tissue sample obtained from patients with a nodal or extra nodal non-cutaneous PTCL, such as angioimmunoblastic T-cell lymphoma (AITL); anaplastic large cell lymphoma, ALK negative; anaplastic large cell lymphoma, ALK positive; enteropathy-associated T-cell lymphoma (EATL); adult T-cell leukaemia/lymphoma (ATLL); extra nodal NK/T cell lymphomas nasal-type; hepatosplenic gamma-delta T-cell lymphomas (HSTL); and peripheral T-cell lymphoma, not otherwise specified (PTCL/NOS) were also assessed for KIR3DL2 overexpression at the surface of T-cells.

The results unambiguously confirmed that T-cells obtained from all 7 Sézary syndrome patients expressed KIR3DL2 at the surface of T-cells (see EXAMPLE 1).

However, and surprisingly, besides Sézary syndrome and transformed mycosis fungoides, a subset of both cutaneous and non-cutaneous nodal and extra nodal PTCL was shown to overexpress KIR3DL2 at the surface of T-cells.

As for cutaneous malignant T cells lymphomas, sub-cutaneous panniculitis-like T-cell lymphoma and primary cutaneous CD8+ aggressive epidermotropic cytotoxic T-cell lymphoma were shown expressing KIR3DL2.

Moreover, both enteropathy-associated T-cell lymphoma and hepatosplenic gamma-delta T-cell lymphoma were shown to represent two non-cutaneous KIR3DL2 expressing malignant T cells lymphomas.

Uses of KIR3DL2 Expression as a Biomarker

Hence, a first aspect of the present invention relates to an in vitro use of a level of expression of KIR3DL2 as a biomarker for diagnosing and/or monitoring a lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma, and hepatosplenic gamma-delta T-cell lymphoma.

A further aspect of the invention relates to an in vitro use of a ratio of levels of expression of KIR3DL2/KIR3DL1 as a biomarker for diagnosing and/or monitoring a lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma, and hepatosplenic gamma-delta T-cell lymphoma.

In one preferred embodiment, a lymphoma is a cutaneous KIR3DL2 expressing malignant T cells lymphoma being selected from the group comprising sub-cutaneous panniculitis-like T-cell lymphoma and primary cutaneous CD8+ aggressive epidermotropic cytotoxic T-cell lymphoma.

In another preferred embodiment, a lymphoma is a non-cutaneous KIR3DL2 expressing malignant T cells lymphoma being selected from the group comprising enteropathy-associated T-cell lymphoma and hepatosplenic gamma-delta T-cell lymphoma.

As used herein, a ratio of levels of expression of KIR3DL2/KIR3DL1 encompasses both (i) a value of the ratio between the measured expression level of KIR3DL2 and the measured expression level of KIR3DL1 and (ii) a value of the ratio between the measured expression level of KIR3DL1 and the measured expression level of KIR3DL2. Preferably herein, a ratio of levels of expression of KIR3DL2/KIR3DL1 consists of a value of the ratio between the measured expression level of KIR3DL2 and the measured expression level of KIR3DL1.

KIR3DL2 is also known as CD158k and NKAT4 gene, or CD158k/KIR3DL2.

KIR3DL1 is also known as CD158e, NKB1, NKAT3 and AMB11, or CD158e/KIR3DL1. For the purpose of the present invention, the inventors consider that these names are equivalent.

KIR3DL1 and KIR3DL2 both refer to the internationally recognized names of the corresponding genes, and proteins in the sequences databases, including the database from the HUGO (Human Genome Organisation) Gene Nomenclature Committee (available notably at http://www.gene.ucl.ac.uk/nomenclature/index.html).

It has been shown that KIR3DL1 was poorly synthesized in T-cells, when mRNA analyses were conducted in healthy individuals and in individuals having a PTCL. A ratio between the respective expression levels of KIR3DL2 and KIR3DL1 thus provides a value that can be directly compared from one individual to another.

In the present invention, the levels of expression of KIR3DL1 and KIR3DL2 are advantageously quantified by measuring the level of mRNA expression.

Any method for measuring the mRNA expression known from the skilled artisan is suitable for implementing the present invention, which includes the well-known RT-PCR method using a specific pair of primers for each target marker.

In another preferred embodiment, the levels of expression of KIR3DL1 and KIR3DL2 are quantified by measuring the level of cellular protein expression, preferably the level of protein surface expression.

In a preferred embodiment, the use according to the present invention further comprises measuring the expression levels of a combination of biomarkers, namely KIR3DL2 and one or more additional biomarkers known to be associated with the said cutaneous lymphomas and the nodal and extra nodal non-cutaneous lymphomas that are within the scope of the present invention. Illustratively, the said one or more additional biomarkers encompass, but are not limited to, the surface membrane complex CD3, the surface membrane proteins CD8, CD30, CD56 and PD1, the CXCL13 chemokine and the cytotoxic proteins granzyme B (GrB) and T-cell intracellular antigen (TiAl), together with the EBV (Epstein-Barr virus) specific EBER (EBV-encoded RNA) transcripts.

According to general embodiments of the present invention described herein, the level of cellular protein expression may be performed notably (i) by measuring the amount of the said protein contained in a whole cell sample or (ii) by measuring the amount of the said protein that is present at the cell surface, preferably at the T-cell surface.

Measuring the amount of a protein marker of interest contained in a whole cell sample may be performed by Western blotting starting from the soluble fraction of a cell lysate and using an antibody directed against the said protein marker of interest, according to methods that are well known by the one skilled in the art.

The expression values of each biomarker of interest, including the expression values of each of KIR3DL2 and KIR3DL1, may be expressed as arbitrary units. Illustratively, the expression value of a biomarker of interest may be expressed as a ratio between (i) the measured expression value of the said biomarker (e.g. KIR3DL2 or KIR3DL1) and (ii) the measured expression value of a gene whose expression level is constant, such as the expression value of CD3δ.

Measuring the amount of a protein marker of interest that is present at the cell surface may be performed by immunochemistry, either by immuno-labelling of fixed cells or by flow immunocytometry, according to methods that are well known by the skilled person in the art.

Thus, in specific embodiment, quantifying the selected markers according to the in vitro diagnosis method described herein encompasses those wherein:
(i) the selected markers are quantified by immunochemical methods, which include quantification of one or more protein markers of interest by immuno-detection methods, for example using antibodies directed specifically against each of the said one or more protein markers, according to well-known immuno-detection methods, for example flow cytometry, and
(ii) the selected markers are quantified by gene expression analysis, which include quantification of one or more marker mRNAs of interest, for example by performing a Real-Time PCR Taqman PCR analysis.

Marker Quantification by Measuring the Level of mRNA Expression

In a preferred embodiment, the said markers are quantified by measuring the level of mRNA expression.

As shown in the examples herein, the KIR3DL2 marker and KIR3DL1 expression may be measured by performing the well-known quantitative real-time PCR (RT-PCR) amplification technique, wherein primers specific for each of the genes KIR3DL2 and KIR3DL1 are used.

In a preferred embodiment, the level of mRNA expression for each of the markers tested is performed using the well-known technique of RT-PCR, then forming complexes between the double-stranded nucleic acids resulting from amplification and fluorescent SYBR® molecules and then by measuring the fluorescence signal generated by the SYBR® molecules complexed with the said amplified nucleic acids.

Primers specific for each of the genes mRNA consists of a routine work for the one skilled in the art. Illustratively, the one skilled in the art may use the specific primers for each of KIR3DL2 and KIR3DL1 that are disclosed in the examples herein.

In some preferred embodiments, quantification of KIR3DL2 may be performed by using the pair of primers of SEQ ID Nº1 and 2:

SEQ ID NO: 1: forward
5′-CAACTTCTCCATCGGTCCCTTGATG-3′;
and
SEQ ID NO: 2: reverse
5′-GTTTGACCACACGCAGGGCAG-3′.

In some preferred embodiments, quantification of KIR3DL1 may be performed by using the pair of primers of SEQ ID Nº3 and 4:

SEQ ID NO: 3: forward
5′-GGACATCGTGGTCACAGGTCC-3′;
and
SEQ ID NO: 4: reverse
5′-GCCTGGAATGTTCTGTTGACCTTGC-3′.

Marker quantification by measuring the level of protein expression

Such techniques include detection and quantification of protein-type markers with any type of ligand molecule that specifically binds thereto, including nucleic acids (for example nucleic acids selected for binding through the well-known SELEX method), antibodies and antibody fragments.

Noticeably, antibodies are presently already available for the biomarker consisting o KIR3DL2 and for KIR3DL1, as described in the present specification.

Illustratively, the one skilled in the art may use the monoclonal anti-KIR3DL2 antibody marketed by the company Aviva Systems Biology, under the Reference number OAAB050807.

Further illustratively, the one skilled in the art may use the monoclonal anti-KIR3DL1 antibody marketed by the company Novus Biologicals under the reference number NPB-147006.

Yet illustratively, the one skilled in the art may use a mouse IgG1 monoclonal antibody that binds to both KIR3DL2 and KIR3DL1. In these embodiments, the expression level value of “KIR3DL2” consists of the expression level value of both KIR3DL2 and KIR3DL1.

As it is shown in the examples herein, there is a low expression level of KIR23DL1 as compared with the expression level of KIR3DL2. This is why measuring only the expression level of KIR3DL2 consists of a relevant biomarker according to the invention.

Further, antibodies to said given marker may be easily obtained with the conventional techniques, including generation of antibody-producing hybridomas.

Hybridomas prepared by conventional techniques are then screened using standard methods to identify one or more hybridomas which produce an antibody which specifically binds with the biological marker protein or a fragment thereof. The invention also encompasses hybridomas made by this method and antibodies made using such hybridomas. Polyclonal antibodies may be used as well.

Thus, in preferred embodiments, expression of a marker is assessed using for example:
a radio-labelled antibody, in particular, a radioactive moiety suitable for the invention may for example be selected within the group comprising ³H, ¹²¹I, ¹²³I, ₉₉mTc, ¹⁴C or ³²P;
a chromophore-labelled or a fluorophore-labelled antibody, wherein a luminescent marker, and in particular a fluorescent marker, suitable for the invention may be any marker commonly used in the field such as fluorescein, BODIPY, fluorescent probes type ALEXA, coumarin and its derivatives, phycoerythrin and its derivatives, or fluorescent proteins such as GFP or the DsRed;
a polymer-backbone-antibody,
an enzyme-labelled antibody, said labelling enzyme suitable for the invention may be an alkaline phosphatase, a tyrosinase, a peroxydase, or a glucosidase; for example, suitable avidin-labelled enzyme may be an avidin-Horse Radish Peroxydase (HRP), and a suitable substrate may be AEC, 5-bromo-4-chloro-3-indolyl phosphate (BCIP), nitro blue tetrazolium chloride (NBT);
an antibody derivative, for example an antibody conjugated with a substrate or with the protein or ligand of a protein-ligand pair, in particular a biotin, a streptavidin or an antibody binding the polyhistidine tag;
an antibody fragment, for example a single-chain antibody, an isolated antibody hypervariable domain, etc., which binds specifically to a marker protein or a fragment thereof, including a marker protein which has undergone all or a portion of its normal post-translational modification.

In vitro techniques for detection of a biological marker protein include enzyme linked immunosorbent assays (ELISAs), Western blots, immunoprecipitations and immunofluorescence.

In another preferred embodiment, the level of expression of KIR3DL2 is expressed as a ratio of levels of expression of KIR3DL2/KIR3DL1.

Method for in Vitro Diagnosing and/or Monitoring

In another aspect, the present invention relates to an in vitro method for diagnosing and/or monitoring a lymphoma in an individual comprising at least a step of quantifying the level of expression of KIR3DL2, said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma, and hepatosplenic gamma-delta T-cell lymphoma.

Within the scope of the instant invention, the methods for in vitro diagnosing and/or monitoring encompass diagnosing and/or monitoring of a cutaneous or a non-cutaneous KIR3DL2 expressing malignant T cells lymphoma.

In one preferred embodiment, a lymphoma is a cutaneous KIR3DL2 expressing malignant T cells lymphoma being selected from the group comprising sub-cutaneous panniculitis-like T-cell lymphoma and primary cutaneous CD8+aggressive epidermotropic cytotoxic T-cell lymphoma.

In another preferred embodiment, a lymphoma is a non-cutaneous KIR3DL2 expressing malignant T cells lymphoma being selected from the group comprising enteropathy-associated T-cell lymphoma and hepatosplenic gamma-delta T-cell lymphoma.

An “individual” to be considered within the present invention may be any subject presenting clinical risks of having a lymphoma, or any subject having been already diagnosed for a lymphoma. Preferably, an individual may be a mammal, and more preferably an animal of economic importance, for example farms, laboratories or food industries animals, such as sheep, swine, cattle, goats, dogs, cats, horses, poultry, mice, rats. Also, an individual according to the invention may be a human. And more preferably, an individual is a human.

The present invention also relates to an in vitro method for diagnosing and/or monitoring a lymphoma in an individual comprising at least a step of quantifying the levels of expression of KIR3DL1 and KIR3DL2, said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma, and hepatosplenic gamma-delta T-cell lymphoma.

The in vitro methods according to the present invention comprise the steps of:
a) providing a biological sample from an individual to be tested,
b) measuring in the said biological sample the expression level of KIR3DL2,
c) diagnosing said lymphoma if the value found at step b) is distinct from a predetermined threshold value for the said expression level and is indicative of a lymphoma positive individual.

In an advantageous embodiment, the method further relies upon quantifying the level of expression of one or more additional biomarkers known to be associated with the said cutaneous lymphomas and the nodal and extra nodal non-cutaneous lymphomas that are within the scope of the present invention, in combination with quantifying the level of expression of KIR3DL2.

Such one or more additional biomarkers may be selected in the group comprising CD3, CD8, CD30, granzyme B and TiAl.

Uses and methods in accordance with the invention are preferably performed with an isolated biological sample. A “biological sample,” as used herein, generally refers to a biological sample obtained, reached, collected or isolated from an individual, in vivo or in situ. Such samples may be, but not limited to, organs, tissues, fractions and cells isolated from a mammal. Exemplary biological samples include but are not limited to a cell culture, a cell line, a tissue biopsy such as a skin biopsy, a nasal tissue biopsy, a gastrointestinal tissue biopsy or lymph node tissue biopsy, an organ, a biological fluid, a blood sample, and the like. Preferred biological samples include but are not limited to a blood sample, peripheral blood mononuclear cells (PBMC) sample or a tissue biopsy, including a skin biopsy, a nasal mucosa biopsy, an intestine biopsy or a lymph node biopsy). The sample can be a crude sample, or can be purified to various degrees prior to storage, processing, or measurement.

An isolated biological sample of the invention comprises T-cells.

In a preferred embodiment, the step of collecting biological samples for the uses and methods of the invention may represent the first step of a use or a method in accordance with the invention.

In another preferred embodiment, the step of collecting biological samples for the uses and methods of the invention is performed before carrying out the invention and is not a step of a use or a method in accordance with the invention.

In one embodiment, an isolated biological sample suitable for the invention comprising T-cells may be selected from the group consisting of a blood sample, a tissue biopsy, a fluid sample.

The samples suitable for the invention can be purified prior to testing. In some embodiments, the blood mononuclear cells, and preferably the T-cells, can be isolated from the remaining cell contents prior to testing. The separating the blood mononuclear cells or the T-cells may be performed by any methods known in the art, for example by density gradient centrifugation.

According to one embodiment, when using an isolated whole blood sample, peripheral blood mononuclear cells (PBMC), comprising lymphocyte cells and monocyte cells, may be separated from plasma (non-cellular components), polynuclear cells, such as neutrophil cells and eosinophil cells, and erythrocytes.

Any known method in the art to separate peripheral blood mononuclear cells (PBMC) from the other blood cell types and non-cellular components may be implemented.

For example, as suitable method, one may cite physical separation methods, such as centrifugations methods. As example of suitable centrifugation methods one may cite gradient density, for example using Ficoll®.

Also one may use immunological separation methods, such as, for example, magnetic beads and flow cytometry.

A threshold value for the specific marker KIR3DL2 or the ratio KIR3DL2/KIR3DL1 may be determined for each specific lymphoma, by carrying out a method comprising the steps of:
a) providing (i) a collection of biological samples from individuals already diagnosed for being positive towards at least one of the subset of cutaneous lymphomas or to nodal and extra nodal non-cutaneous lymphomas within the scope of the present invention and (ii) a collection of biological samples from individuals diagnosed for being negative towards said lymphomas,
b) quantifying for each sample from collection (i) provided at step a) the level of expression of KIR3DL2, and optionally KIR3DL1 (to obtain a ratio KIR3DL2/KIR3DL1), whereby a first collection of quantification values for the said marker(s) is obtained,
c) quantifying for each sample from collection (ii) provided at step a) the level of expression of KIR3DL2, and optionally KIR3DL1 (to obtain a ratio KIR3DL2/KIR3DL1), whereby a second collection of quantification values for the said marker(s) is obtained,
d) calculating, from the said first collection of quantification values obtained at the end of step b), the mean quantification value for the said marker in lymphoma-negative individuals,
e) calculating, from the said second collection of quantification values obtained at the end of step b), the mean quantification value for the said marker in one of the said lymphoma-positive individuals,
f) calculating, a threshold value that optimally discriminates between said lymphoma-positive and lymphoma-negative individuals from the mean quantification values obtained at steps d) and e), respectively.

It has to be understood that the lymphoma-positive individuals from step f) of the above-described method is intended to be selected in the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

Furthermore and for the sake of clarity, the expression « optimally discriminates » that is used for describing step f) of the method above means that the said threshold value is calculated and the said value lies between (i) the mean quantification value that is obtained at step d) and the mean quantification value that is obtained at step e) and is the most discriminating between lymphoma-positive and lymphoma-negative individuals.

A threshold value described above shall be performed for each lymphoma in the in vitro diagnosis method of the invention, for the purpose of performing a reliable diagnosis of a lymphoma, from a subset of lymphoma candidates, in an individual.

The diagnosis methods from the present invention are intended to provide a first approach to discriminate a subset of specifically defined cutaneous lymphomas and nodal and extra nodal non-cutaneous lymphomas, from the bulk of the PTCL diseases.

Indeed, the threshold values that may be used when performing the in vitro diagnosis method disclosed herein may be expressed as arbitrary units that reflect the expression level of the KIR3DL2 marker in the analysed biological sample, the said expression level either consisting of a protein expression level, for example a cell surface expression level, or a gene expression level, for example a mRNA expression level.

In some embodiments, the in vitro methods according to the present invention comprise the steps of:
a) providing a biological sample from an individual to be tested,
b) measuring in the said biological sample the ratio of levels of expression of KIR3DL2/KIR3DL1,
c) diagnosing said lymphoma if the value found at step b) is distinct from a predetermined threshold value for the said ratio is indicative of a lymphoma positive individual.

In a preferred embodiment, the method further encompasses quantifying the level of expression of one or more additional biomarkers already known to be associated with the said cutaneous lymphomas and the nodal and extra nodal non-cutaneous lymphomas that are within the scope of the present invention.

Another aspect of the invention relates to a method for monitoring the effectiveness of treatment against a lymphoma, in an individual in need thereof, with a therapeutic agent, said method comprising the steps of:
(i) providing a pre-administration biological sample from an individual prior to administration of the therapeutic agent;
(ii) measuring the level of expression of KIR3DL2 in the pre-administration biological sample;
(iii) providing one or more post-administration biological samples from the individual;
(iv) measuring the level of expression of KIR3DL2 in the post-administration biological samples;
(v) comparing the level of expression of KIR3DL2 in the pre-administration biological sample with the level of expression of KIR3DL2 in the post-administration biological sample or samples; and
(vi) altering the administration of the therapeutic agent to the individual accordingly, said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In a preferred embodiment, the invention relates to a method for monitoring the effectiveness of treatment against a lymphoma, in an individual in need thereof, with a therapeutic agent, said method comprising the steps of:
(i) providing a pre-administration biological sample from an individual prior to administration of the therapeutic agent;
(ii) measuring the ratio of the levels of expression of KIR3DL2/KIR3DL1 in the pre-administration biological sample;
(iii) providing one or more post-administration biological samples from the individual;
(iv) measuring the ratio of the levels of expression of KIR3DL2/KIR3DL1 in the post-administration biological samples;
(v) comparing the ratio of the levels of expression of KIR3DL2/KIR3DL1 in the pre-administration biological sample with the ratio of the levels of expression of KIR3DL2/KIR3DL1 in the post-administration biological sample or samples; and
(vi) altering the administration of the therapeutic agent to the individual accordingly, said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

Advantageously, a method for monitoring the effectiveness of treatment against a lymphoma within the scope of the present invention may comprise measuring the level of one or more additional bio markers that have been already been identified in the art to be specific for each kind of lymphoma.

For example, a worse diagnosis that is determined by assessing the expression level of the KIR3DL2 biomarker or the KIR3DL2/KIR3DL1 ratio, during the course of treatment may indicate ineffective dosage and the desirability of increasing the dosage. Conversely, a better diagnosis that is determined by assessing the expression level of the selected markers, namely the level of expression of KIR3DL2 or the ratio KIR3DL2/KIR3DL1, may indicate efficient treatment and hence the absence of a need to change dosage.

The present invention also relates to a method for adapting a treatment against a lymphoma in an individual in need thereof, wherein said method comprises at least the steps of:
a) performing, on at least one biological sample collected from said individual, the in vitro diagnosis method according to the present invention; and
b) adapting the treatment against the lymphoma of said individual by administering to said individual a suitable therapy,
said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

A suitable therapy may include chemotherapy, radiotherapy and bone marrow transplantation.

As examples of chemotherapy suitable for treating the cutaneous lymphomas and the nodal and extra nodal non-cutaneous lymphomas within the scope of the present invention one may cite cyclophosphamide, doxorubicin, vincristine, prednisone, etoposide, ifosfamide, carboplatin, gemcitabine, vinorelbine, dexamethasone, cytarabine, cisplatin and the like.

Methods for Screening Compounds of Therapeutic Interest

As an increase of KIRD3DL2 expression level is specifically correlated with a subset of cutaneous and nodal and extra nodal non-cutaneous lymphomas, isolating, screening and administering compounds that affect KIR3DL2 expression levels and/or biological activities may be useful in treating and/or preventing the occurrence of such hyper-proliferative T-cell lymphomas. Compounds of interest are especially those which induce an inhibition of the expression of KIR3DL2 in T cells.

Hence, another aspect of the present invention relates to a method for screening a compound candidate that affects KIR3DL2 expression level, said method comprising the step of:
a) providing at least one T-cell able to express KIR3DL2;
b) measuring KIR3DL2 expression level by the at least one T-cell provided at step a), whereby a first KIR3DL2 expression value is obtained;
c) incubating the said KIR3DL2 expressing at least one T-cell with a candidate compound to be tested;
d) measuring the KIR3DL2 expression level by the KIR3DL2 expressing at least one T-cell of step c), whereby a second KIR3DL2 expression value is obtained;
e) comparing the said first KIR3DL2 expression value with the said second KIR3DL2 expression value. and
f) selecting the said candidate compound when the said second KIR3DL2 expression value is lower than the said first KIR3DL2 expression value.

Alternatively, both KIR3DL1 and KIR3DL2 expression levels may be measured at steps b) and d) of the above described screening method, and the ratios KIR3DL2/KIR3DL1 corresponding to the first and the second values are calculated at the end of steps b) and d) respectively and compared at the end of step e).

In a preferred embodiment, the second KIR3DL2 expression value is lowered as compared to the quantitative first value by a factor at least of about 2, for example of about 3, for example of about 4, for example of about 5, for example of about 10, for example of about 20 or for example of about 50.

In some embodiments, candidate compounds encompass small organic molecules that may be obtained either after purification from a natural source or after semi- or whole chemical synthesis. In addition to the above described compounds aimed for inhibiting the gene expression of KIR3DL2, it is envisioned that small molecules or other natural products may be identified and employed to inhibit the transcription in vivo of the KIR3DL2 gene.

In some embodiments, candidate compounds encompass a ribozyme, an antisense oligonucleotide, a triple helix DNA, a RNA aptamer and/or double-stranded RNA directed to an appropriate nucleotide sequence of KIR3DL2 nucleic acid. These compounds may be identified, isolated or synthesized de novo, using conventional techniques known from a skilled person in the art without undue burden or experimentation. For example, inhibition of KIR3DL2 gene expression can be obtained by designing antisense molecules, of DNA- or RNA-type, targeted towards the important regions of the gene encoding the KIR3DL2 protein.

In another aspect, the present invention relates to a method for the screening of a candidate compound that affects KIR3DL2 biological activity, said method comprising the step of:
a) providing at least one T-cell able to express KIR3DL2;
b) measuring KIR3DL2 biological activity in the at least one T-cell provided at step a), whereby a first activity value is obtained;
c) incubating KIR3DL2 expressing T-cell with a candidate compound to be tested;
d) measuring the KIR3DL2 biological activity in the KIR3DL2 expressing T-cell obtained at the end of step b), whereby a second activity value is obtained;
e) comparing the said first activity value with the said second activity value.

In preferred embodiments, the said candidate compound is selected at a further step f), when the said second activity value is lower than the said first activity value.

Advantageously, the second value is lowered as compared to the quantitative first value by a factor at least of about 2, for example of about 3, for example of about 4, for example of about 5, for example of about 10, for example of about 20 or for example of about 50.

In the method described above, the biological activity represents, but is not limited to, KIR3DL2 localization at the membrane compartment or KIR3DL2 ability to bind and/or interact with its cellular and/or extracellular partners molecules.

In another aspect, the invention relates to a method for treating or ameliorating a condition of an individual having one of the lymphoma selected within the subset of cutaneous and nodal and extra nodal non-cutaneous lymphoma herein described, comprising administering to said individual in need thereof a pharmaceutical composition comprising an effective amount of at least a compound that affects KIR3DL2 expression levels and/or biological activity, and most preferably of at least a compound that inhibits KIR3DL2 expression levels, or biological activity.

In another preferred embodiment, a compound that affects KIR3DL2 biological activity encompasses antagonists directed towards the KIR3DL2 protein activity.

The decreased biological activity, which is aimed as a consequence of the antagonist administration, may be caused by, but is not limited to, a decrease of KIR3DL2 amount in the cellular environment, a defect of KIR3DL2 localization at the membrane compartment, a decrease of KIR3DL2 ability to bind and/or interact with its cellular and/or extracellular partner molecules.

As it is used herein, the term “antagonist” refers to a molecule which decreases the biological activity of KIR3DL2. Antagonists can include, but are not limited to, peptides, proteins, nucleic acids (DNA- and RNA-type aptamers), carbohydrates, antibodies or any molecules which decrease the amount or the biological activity of the KIR3DL2 protein.

In a preferred embodiment, the antagonist is an antibody, or an active fragment thereof, such as Fab, F(ab)2, Fab′, F(ab′)2, Fv and the like that are capable of binding an epitopic determinant, which is involved in said biological activity.

Antibodies or active fragment thereof can be prepared according to the well-known methods available to the skilled person in the art.

The inhibitory compounds encompassed by the present invention can be administered as pharmaceutical compositions. Such pharmaceutical compositions for use in accordance with the present invention may be formulated in a conventional manner using one or more physiologically acceptable carriers or excipients.

Thus, the compounds and their physiologically acceptable salts and solvates may be formulated for any possible route of administration, including but not limited to topical, oral, buccal, systemic, parenteral or rectal administration.

Kits

Another aspect of the present invention relates to a kit for diagnosing and/or monitoring a lymphoma in an individual, which kit comprises means for quantifying the level of expression of KIR3DL2 or alternatively the levels of expression of KIR3DL1 and KIR3DL2, said lymphoma being selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In a preferred embodiment, the kit further comprises means for quantifying the level of expression of one or more additional biomarkers already correlated with the said cutaneous lymphomas and the nodal and extra nodal non-cutaneous lymphomas that are within the scope of the present invention.

Hence, in one preferred embodiment. the present invention relates to a kit for diagnosing and/or monitoring a cutaneous KIR3DL2 expressing malignant T cells lymphoma in an individual, which kit comprises means for quantifying the level of expression of KIR3DL2 or alternatively the levels of expression of KIR3DL1 and KIR3DL2, said lymphoma being selected from the group comprising sub-cutaneous panniculitis-like T-cell lymphoma and primary cutaneous CD8+ aggressive epidermotropic cytotoxic T-cell lymphoma.

In another preferred embodiment, the present invention relates to a kit for diagnosing and/or monitoring a non-cutaneous KIR3DL2 expressing malignant T cells lymphoma in an individual, which kit comprises means for quantifying the level of expression of KIR3DL2 or alternatively the levels of expression of KIR3DL1 and KIR3DL2, said lymphoma being selected from the group comprising enteropathy-associated T-cell lymphoma and hepatosplenic gamma-delta T-cell lymphoma.

In a still preferred embodiment, one may take advantage of additional biomarkers, which have been shown to be correlated with one or more of the lymphoma(s) encompassed by the present invention. Such additional biomarkers are not limited to CD3, CD8, CD30, CD56, PD1, CXCL13, granzyme B, TiAl.

Suitable reagents for binding with a marker nucleic acid (e.g. a genomic DNA, an mRNA, a spliced mRNA, a cDNA, or the like) include complementary nucleic acids. For example, the nucleic acid reagents may include oligonucleotides (labelled or non-labelled) fixed to a substrate, labelled oligonucleotides not bound with a substrate, pairs of PCR primers, molecular beacon probes, and the like.

Advantageously, the kit according to the present invention enables to quantify the level of expression KIR3DL2 and/or KIR3DL1 by measuring the level of mRNA expression.

In a preferred embodiment, the kit comprises at least a set of 2 primers that hybridize specifically to a portion of KIR3DL2 or KIR3DL1 mRNAs. These primers enable a skilled artisan to implement the RT-PCR technique.

In a still preferred embodiment, the kit according to the present invention enables to quantify the level of expression of KIR3DL2 and/or KIR3DL1 by measuring the level of cellular protein expression, preferably the level of protein surface expression.

Protein expression may be quantified by specific antibodies. Yet, suitable antibodies for the invention may be a polyclonal or monoclonal type IgG, IgA, IgM, or IgE. An antibody suitable for the invention may be selected from antibodies from mouse, rat, rabbit, goat, horse, llama, human or other primate.

An antibody fragment having binding properties defined above may also be suitable for the invention. By “antibody fragment” is meant a portion of an antibody such as Fab, Fab′, F(ab)2, F(ab′)2 fragments and other similar. These terms also include any synthetic or genetically engineered protein that can act as an antibody by binding to a detectable protein of the invention, in a protein complex as defined above.

An antibody or antibody fragment suitable for the invention may be prepared by any method known to those skilled in the art, as described, for example, in “Making and using antibodies: a practical handbook” (Howard & Kaser, Ed CRC, 2006).

The kit may comprise a plurality of reagents, each of which is capable of binding specifically with the nucleic acid marker or the protein marker KIR3DL2 and optionally the nucleic acid or the protein KIR3DL1.

Suitable reagents for binding with a marker protein include antibodies, antibody derivatives, antibody fragments, and the like.

Thus, a further object of this invention consists of a kit for the diagnosis of the occurrence of a subset of cutaneous and nodal and extra nodal non-cutaneous lymphomas, which kit comprises means for quantifying at least one marker, i.e. KIR3DL2 and optionally KIR3DL1.

The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of the in vitro diagnosis method of the invention, and the like.

Alternatively, the kit described above may be useful for screening a compound candidate that affects KIR3DL2 expression level and/or biological activity, as described above.

The examples presented hereafter are for illustrating purpose of the invention and should not be construed as limiting the scope of the invention.

KIR3DL2 as a Therapeutic Target

KIR3DL2 has been reported to be considered as a valuable target for Sézary syndrome therapy.

Indeed, a monoclonal antibody specifically targeted against KIR3DL2 was shown to inhibit cellular proliferation and to further promote specific cell death of the KIR3DL2 expressing malignant T-cells, by a mechanism involving antibody-dependent cellular cytotoxicity (ADCC) (WO 2010/081890).

Moreover, several oligodeoxynucleosides that are rich in CpG dinucleotides (CpG ODN) were shown to bind to KIR3DL2 from both T-cells from Sézary individuals and NK cells (Natural Killer lymphocytes), and may elicit cytokines release from KIR3DL2 expressing NK cells, as disclosed in Sivori et al. (A novel KIR-associated function: evidence that CpG DNA uptake and shuttling to early endosomes is mediated by KIR3DL2; Blood, 2010, 116(10):1637-1647).

CpG ODN have been reported to induce tumor regression by activating innate immunity, to enhance antigen-dependent cellular cytotoxicity (ADCC), and to be a valuable vaccine adjuvant that elicit a specific, protective immune response, and to be also good candidates for the treatment of various types of cancerous and non-cancerous diseases (Bodera et al. Synthetic immunostimulatory oligonucleotides in experimental and clinical practice. Pharmacol Rep. 2012 Sep;64(5):1003-10).

Furthermore, GpC ODN (oligonucleotides that are rich in GpC dinucleotides), often used as controls ODN, were also tested.

Compounds or Pharmaceutical Composition for KIR3DL2+ Lymphomas Treatment

The inventors' findings disclosed herein according to which a plurality of specific T-cell lymphomas express KIR3DL2 at the T-cell surface has allowed them to design novel therapeutic tools for treating these specific T-cell lymphomas.

More precisely, the inventors' findings disclosed herein have allowed designing therapeutic tools that are ligands binding to KIR3DL2 and wherein the binding event of the said ligands to KIR3DL2 causes the death of the malignant T-cells.

In another aspect, the present invention describes a ligand molecule that specifically binds to KIR3DL2 for the prevention and/or the treatment of a KIR3DL2+ lymphoma selected from the group comprising Sezary syndrome, transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

In another aspect, the present invention relates to a ligand molecule that specifically binds to KIR3DL2 for the prevention and/or the treatment of a KIR3DL2+ lymphoma selected from the group comprising transformed mycosis fungoides, sub-cutaneous panniculitis-like T-cell lymphoma, primary cutaneous CD8+aggressive epidermotropic cytotoxic T-cell lymphoma, enteropathy-associated T-cell lymphoma, adult T-cell leukaemia/lymphoma and hepatosplenic gamma-delta T-cell lymphoma.

In another aspect, the present invention relates to a ligand molecule that specifically binds to KIR3DL2 for the prevention and/or the treatment of a KIR3DL2+ lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In a still another aspect, the present invention relates to a ligand molecule that specifically binds to KIR3DL2 for the prevention and/or the treatment of a cutaneous KIR3DL2+ lymphoma selected from the group comprising sub-cutaneous panniculitis-like T-cell lymphoma and primary cutaneous CD8+ aggressive epidermotropic cytotoxic T-cell lymphoma.

In another aspect, the present invention relates to a ligand molecule that specifically binds to KIR3DL2 for the prevention and/or the treatment of a non-cutaneous KIR3DL2+ lymphoma selected from the group comprising enteropathy-associated T-cell lymphoma and hepatosplenic gamma-delta T-cell lymphoma.

The ligand molecule according to the present invention is capable of specifically inducing the death of KIR3DL2 expressing malignant T-cells. In particular, the death of KIR3DL2 expressing malignant T-cells is mediated by a process selected from the group comprising apoptosis, antigen-dependent cellular cytotoxicity (ADCC) and complement-dependent cytotoxicity (CDC).

The ligand molecule according to the present invention is selected from the group comprising an antibody, a fragment of an antibody and an oligodeoxynucleotide.

In a preferred embodiment the present invention relates to an anti-KIR3DL2 antibody that specifically binds to KIR3DL2 for the prevention and/or the treatment of a KIR3DL2+ lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma and hepatosplenic gamma-delta T-cell lymphoma.

In another preferred embodiment the present invention relates to an anti-KIR3DL2 antibody that specifically binds to KIR3DL2 for the prevention and/or the treatment of a cutaneous KIR3DL2+ lymphoma selected from the group comprising sub-cutaneous panniculitis-like T-cell lymphoma and primary cutaneous CD8+ aggressive epidermotropic cytotoxic T-cell lymphoma.

In a still another preferred embodiment the present invention relates to an anti-KIR3DL2 antibody that specifically binds to KIR3DL2 for the prevention and/or the treatment of a non-cutaneous KIR3DL2+ lymphoma selected from the group comprising enteropathy-associated T-cell lymphoma and hepatosplenic gamma-delta T-cell lymphoma.

Anti-KIR3DL2 antibodies within the scope of the present invention can be obtained according to methods known from a skilled person in the art, such as, for example, the hybridoma method. Various adjuvants known in the art can be employed to enhance antibody production.

Anti-KIR3DL2 antibodies may be polyclonal, although monoclonal antibodies are preferred.

A skilled person in the art can easily select anti-KIR3DL2 antibodies suitable to deplete malignant T-cells expressing KIR3DL2 at their surface, for example anti-KIR3DL2 antibodies that deplete malignant KIR3DL2 expressing T-cells via antibody-dependent cell mediated cytotoxicity (ADCC), complement dependent cytotoxicity (CDC), inhibition of cell proliferation or induction of cell death (e.g. via apoptosis).

Antigen-dependent cellular cytotoxicity (ADCC) may be assessed according to the protocol disclosed by, for example, Nelson et al. (⁵¹Cr release assay of antibody-dependent cell-mediated cytotoxicity (ADCC). Curr Protoc Immunol. 2001 May; Chapter 7: Unit 7.27); Broussas et al. (Evaluation of antibody-dependent cell cytotoxicity using lactate dehydrogenase (LDH) measurement. Methods Mol Biol. 2013; 988:305-17.

Complement-dependent cytotoxicity (CDC) may be assessed according to the protocol disclosed by, for example, Harmer et al. (A highly sensitive, rapid screening method for the detection of antibodies directed against HLA class I and class II antigens. Transpl Int 1993; 6:277-80); Robson et al. (A comparison of flow cytometry screening methods. Eur J Immunogenetics 1999; 26:43-80); Broyer et al. (Evaluation of complement-dependent cytotoxicity using ATP measurement and C1q/C4b binding. Methods Mol Biol. 2013; 988:319-29).

Apoptosis may be assessed by numerous protocols or kits well known from the skilled person in the art.

For example, apoptosis may be assessed by assaying caspase induced activity, for example by using one of the commercially available kits such as Caspase 3 Activity Assay (Roche Applied Science), Apo-ONE® Homogeneous Caspase-3/7 Assay (Promega), EnzChek® Caspase-3 Assay Kit #1 (Invitrogen), following the manufacturer's instructions.

Apoptosis may also be assessed by assaying tunel and DNA fragmentation, for example by using one of the commercially available kits such as Apoptotic DNA Ladder Kit (Roche Applied Science), DeadEndυ Fluorometric TUNEL System (Promega), APO-BrdU™ TUNEL Assay Kit (Invitrogen), Apoptotic DNA Ladder Kit (Genotech), following the manufacturer's instructions.

Other methods are available to assess apoptosis, for example measuring cell permeability, staining phosphatisylserine by Annexin V, measuring mitochondrial membrane potential, etc.

Advantageously, the ability of the ligand molecule, within the scope of the instant invention, to elicit the death of KIR3DL2 expressing malignant T-cells may be assessed in vitro on an isolated cell line.

As an illustrative example for a Sézary syndrome cell line available for such an assay, also non limiting, one can mention HUT-78 (available at the American Type Culture Collection (ATCC), as ATCC T1B-161); HH (ATCC CRL-2105), SeAx (Kaltoft et al. A continuous T-cell line from a patient with Sézary syndrome. Arch Dermatol Res. 1987; 279(5):293-8.); MyLa 2059 (Kaltoft, University of Aahrus, Denmark); P1 (Marie-Cardine et al. Killer cell Ig-like receptors CD158a and CD158b display a coactivatory function, involving the c-Jun NH2-terminal protein kinase signaling pathway, when expressed on malignant CD4+ T cells from a patient with Sezary syndrome. Blood 2007; 109: 5064-5); PNO (Poszepczynska et al. Functional characterization of an IL-7-dependent CD4(+)CD8alphaalpha(+) Th3-type malignant cell line derived from a patient with a cutaneous T-cell lymphoma. Blood 2000; 96:1056-1063).

Anti-KIR3DL2 antibodies, which include humanized antibodies, and antibody fragments thereof, may be prepared according to known techniques. In a particular embodiment, the anti-KIR3DL2 antibody is a chimeric, a humanized or a full-human anti-KIR3DL2 antibody.

In another particular embodiment, the anti-KIR3DL2 antibody is an antibody fragment selected from the group of F(ab′)2, F(ab)2, Fab′, Fab, Fv, scFv, i.e. a fragment bearing the minimal recognition moieties.

In a still preferred embodiment, the anti-KIR3DL2 antibody is a monoclonal antibody selected from the group consisting of a human antibody, a humanized antibody, and a chimeric antibody.

Preferably, the anti-KIR3DL2 antibody induces antibody-dependent cellular cytotoxicity (ADCC). In certain embodiments, the anti-KIR3DL2 antibody is an IgG1 or IgG3 human isotype antibody. In other embodiments, the anti-KIR3DL2 antibody is an IgG2 or IgG4 human isotype antibody.

In another preferred embodiment, the anti-KIR3DL2 antibody induces complement cell toxicity mechanism (CDC).

In another preferred embodiment, the anti-KIR3DL2 antibody induces apoptosis.

In a preferred embodiment, the ligand molecule is the AZ158 monoclonal antibody mAb, as previously described in Parolini et al. (The AZ158 mAb specifically reacts with p70 and p140 inhibitory NK receptors for HLA-B and HLA-A alleles. Leukocyte Typing VII. 2002. In: (Mason D, Andre P, Bensussan A, Buckley C, Civin C, Clark E et al., eds) Oxford: Oxford University Press, 415-417).

In a preferred embodiment, the ligand molecule is the Q66 monoclonal antibody (Pende et al. The natural killer cell receptor specific for HLA-A allotypes: a novel member of the p58/p70 family of inhibitory receptors that is characterized by three immunoglobulin-like domains and is expressed as a 140-kD disulphide-linked dimer. J Exp Med. 1996;184:505-18).

In a preferred embodiment, the ligand molecule according to the instant invention is selected from the group comprising AZ158 and Q66 monoclonal antibodies.

In another aspect, the present invention describes an oligodeoxynucleotide that specifically binds to KIR3DL2 for the prevention and/or the treatment of a KIR3DL2+ lymphoma selected from the group comprising Sézary syndrome, transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

In one aspect, the present invention relates to an oligodeoxynucleotide that specifically binds to KIR3DL2 for the prevention and/or the treatment of a KIR3DL2+ lymphoma selected from the group comprising transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

In one aspect, the present invention relates to an oligodeoxynucleotide that specifically binds to KIR3DL2 for the prevention and/or the treatment of a KIR3DL2+ lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

In another aspect, the present invention relates to an oligodeoxynucleotide that specifically binds to KIR3DL2 for the prevention and/or the treatment of a cutaneous KIR3DL2+ lymphoma selected from the group comprising sub-cutaneous panniculitis-like T-cell lymphoma and primary cutaneous CD8+ aggressive epidermotropic cytotoxic T-cell lymphoma.

In another aspect, the present invention relates to an oligodeoxynucleotide that specifically binds to KIR3DL2 for the prevention and/or the treatment of a non-cutaneous KIR3DL2+ lymphoma selected from the group comprising enteropathy-associated T-cell lymphoma, adult T-cell leukaemia/lymphoma and hepatosplenic gamma-delta T-cell lymphoma.

It is shown in the examples herein that CpG ODN treatment of T-cells from Sézary individuals results in (i) the binding of CpG ODN to the KIR3DL2 at the surface of the T-cells; (ii) the internalization of KIR3DL2, hence its depletion from the surface of the T-cells; and unexpectedly (iii) the induction of a caspase-dependent apoptotic pathway.

Documents WO 01/22972 and EP 2 290 078 may be mentioned as relating to the use of CpG ODN for their immunostimulatory functions, and notably for treating various type of cancers.

Advantageously, the ligand molecule according to the instant invention may be an oligodeoxynucleotide selected from the group comprising CpG ODN-A of sequence SEQ ID NO: 5; CpG ODN-B of sequence SEQ ID NO: 6; CpG ODN-C of sequence SEQ ID NO: 7, mixtures thereof and/or analogs thereof.

Analogs of the oligodeoxynucleotides selected from the group comprising CpG ODN-A of sequence SEQ ID NO: 5; CpG ODN-B of sequence SEQ ID NO: 6; CpG ODN-C of sequence SEQ ID NO: 7 comprise oligonucleotides with a nucleotide sequence at least 50% identical, for example at least 60% identical, at least 70% identical, at least 80% identical, at least 90% identical, at least 95% identical, to either SEQ ID NO: 5, SEQ ID NO: 6 or SEQ ID NO: 7.

In a preferred embodiment, the ligand molecule according to the instant invention may be the oligodeoxynucleotide CpG ODN-C of sequence SEQ ID NO: 7.

Surprisingly, when GpC ODN, often used as a CpG ODN negative control, was used to assess for KIR3DL2 expressing malignant T-cells treatment for inducing apoptosis, it was found to be as active as the CpG ODN-C.

Hence, in another embodiment of the instant invention, GpC ODN is a ligand molecule inducing KIR3DL2 expressing malignant T-cells apoptosis.

In a preferred embodiment, GpC ODN of sequence SEQ ID NO: 8 may be used as a ligand molecule to treat KIR3DL2 expressing malignant T-cells.

In another further embodiment, analogs of the oligodeoxynucleotides GpC ODN of sequence SEQ ID NO: 8, comprising oligonucleotides with a nucleotide sequence at least 50% identical, for example at least 60% identical, at least 70% identical, at least 80% identical, at least 90% identical, at least 95% identical to SEQ ID NO: 8 may be used as a ligand molecule to treat KIR3DL2 expressing malignant T-cells.

More advantageously, the ligand molecule according to the instant invention may thus be an oligodeoxynucleotide selected from the group comprising CpG ODN-A of sequence SEQ ID NO: 5; CpG ODN-B of sequence SEQ ID NO: 6; CpG ODN-C of sequence SEQ ID NO: 7; GpC ODN of sequence SEQ ID NO: 8, mixtures thereof and/or analogs thereof

In a still preferred embodiment, an anti-KIR3DL2 antibody is in a mixture with an oligonucleotide, or an analog thereof, as to potentiate the effects of both ligand molecules.

In a preferred embodiment, the ligand molecule within the scope of the instant invention is capable of inducing at least about 10%, for example about 20%, for example about 30%, for example about 40%, for example about 50%, for example about 60%, for example about 70%, for example about 80%, for example about 90% of cell death, as assessed in a cytotoxic assay.

A pharmaceutical composition comprising a ligand molecule, as defined in the instant invention, and a pharmaceutically acceptable carrier for the prevention and/or the treatment of a KIR3DL2+ lymphoma selected from the group comprising sub-cutaneous panniculitis-like T-cell lymphoma, primary cutaneous CD8+ aggressive epidermotropic cytotoxic T-cell lymphoma, enteropathy-associated T-cell lymphoma, and hepatosplenic gamma-delta T-cell lymphoma.

Furthermore, the present invention also relates to a ligand molecule that specifically binds to the extracellular domain of KIR3DL2 and is able to induce the cell death of the malignant T-cells, for the prevention and/or treatment of a subset of cutaneous lymphomas and a subset of non-cutaneous nodal and extra nodal lymphomas.

The invention further results from the discovery that ligand molecules that bind KIR3DL2 receptor, and in particular the extracellular domain of KIR3DL2 receptor, are capable to induce a decrease of the proliferation of KIR3DL2-expressing malignant T cells, i.e. by inducing a KIR3DL2-mediated inhibitory signal.

In some embodiments, therapeutic compositions and regimens are herein disclosed and used for treating individuals previously diagnosed with KIR3DL2 expressing malignant T-cells lymphomas such as sub-cutaneous panniculitis-like T-cell lymphoma, primary cutaneous CD8+ aggressive epidermotropic cytotoxic T-cell lymphoma, enteropathy-associated T-cell lymphoma, and hepatosplenic gamma-delta T-cell lymphoma.

Methods for Treating a KIR3DL2 Expressing Malignant T-cells Lymphoma

A further aspect of the invention relates to a method for treating a KIR3DL2 expressing malignant T-cells lymphoma in an individual in need thereof, comprising administering to said individual a ligand molecule that specifically binds to KIR3DL2.

In particular, said KIR3DL2 expressing malignant T-cells lymphoma is selected from the group comprising transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

A further aspect of the invention relates to a method for treating a cutaneous KIR3DL2 expressing malignant T-cells lymphoma in an individual in need thereof, comprising administering to said individual a ligand molecule that specifically binds to KIR3DL2.

In particular, said cutaneous KIR3DL2 expressing malignant T-cells lymphoma is selected from the group comprising transformed sub-cutaneous panniculitis-like T-cell lymphoma and primary cutaneous CD8+ aggressive epidermotropic cytotoxic T-cell lymphoma.

A further aspect of the invention relates to a method for treating a non-cutaneous KIR3DL2 expressing malignant T-cells lymphoma in an individual in need thereof, comprising administering to said individual a ligand molecule that specifically binds to KIR3DL2.

In particular, said non-cutaneous KIR3DL2 expressing malignant T-cells lymphoma is selected from the group comprising enteropathy-associated T-cell lymphoma, adult T-cell leukaemia/lymphoma and hepatosplenic gamma-delta T-cell lymphoma.

In another aspect, the present invention relates to a method for treating a KIR3DL2 expressing malignant T-cells lymphoma in an individual in need thereof, comprising administering to said individual a pharmaceutical composition comprising a ligand molecule that specifically binds to KIR3DL2, and a pharmaceutically acceptable carrier.

In a preferred embodiment, the present invention relates to a method for treating a KIR3DL2 expressing malignant T-cells lymphoma in an individual in need thereof, comprising administering to said individual a pharmaceutical composition comprising a ligand molecule that specifically binds to KIR3DL2, and a pharmaceutically acceptable carrier, wherein said lymphoma is selected from the group comprising transformed mycosis fungoides, sub-cutaneous panniculitis-like T-cell lymphoma, primary cutaneous CD8+ aggressive epidermotropic cytotoxic T-cell lymphoma, enteropathy-associated T-cell lymphoma, adult T-cell leukaemia/lymphoma and hepatosplenic gamma-delta T-cell lymphoma.

In another preferred embodiment, the present invention relates to a method for treating a cutaneous KIR3DL2 expressing malignant T-cells lymphoma in an individual in need thereof, comprising administering to said individual a pharmaceutical composition comprising a ligand molecule that specifically binds to KIR3DL2, and a pharmaceutically acceptable carrier, wherein said lymphoma is selected from the group comprising sub-cutaneous panniculitis-like T-cell lymphoma and primary cutaneous CD8+ aggressive epidermotropic cytotoxic T-cell lymphoma.

In a still another preferred embodiment, the present invention relates to a method for treating a non-cutaneous KIR3DL2 expressing malignant T-cells lymphoma in an individual in need thereof, comprising administering to said individual a pharmaceutical composition comprising a ligand molecule that specifically binds to KIR3DL2, and a pharmaceutically acceptable carrier, wherein said lymphoma is selected from the group comprising enteropathy-associated T-cell lymphoma, adult T-cell leukaemia/lymphoma and hepatosplenic gamma-delta T-cell lymphoma.

In a preferred embodiment, the present invention concerns a method for treating a KIR3DL2 expressing malignant T-cells lymphoma in an individual in need thereof, comprising administering to said individual a ligand molecule that specifically binds to KIR3DL2, in an amount sufficient to deplete T-cells.

In a preferred embodiment, said ligand molecule that binds specifically to KIR3DL2 depletes circulating and/or organ-localized malignant T-cells.

In a preferred embodiment, the dosage regimen of the ligand molecule or the pharmaceutical composition disclosed herein is established by a physician. The specific therapeutically effective dosage regimen, and the amount sufficient to deplete T-cells, for a particular individual in need of the treatment will be dependent upon a variety of factors including, but not limited to: the T-cell lymphoma being treated and the severity of the disorder; the age; the body weight; general health; the sex; the diet; the time course of administration; the route of administration; the duration of the treatment; the drugs that are concomitantly administered in combination with the ligand molecule or pharmaceutical composition within the scope of the present invention.

In a most preferred embodiment, the dosage regimen of the ligand molecule or the pharmaceutical composition herein disclosed may range from about 0.01 to about 1,000 mg per adult per day. Preferably, the patient is administered with an amount of about 0.01, 0.05, 0.1, 0.5, 1.0, 2.5, 5.0, 10.0, 15.0, 25.0, 50.0, 100, 250 and 500 mg of the ligand molecule in order to adjust the dosage regimen that is the most suitable to a particular individual in need of the treatment. A pharmaceutical composition within the scope of the present invention may contain from about 0.01 mg to about 500 mg of the ligand molecule, preferably from about 1 mg to about 100 mg of the ligand molecule.

In a preferred embodiment, an effective amount of the ligand molecule is routinely administered at a dosage regimen from about 0.0002 mg/kg to about 20 mg/kg of body weight per day, especially from about 0.001 mg/kg to 7 mg/kg of body weight per day.

The optimal amount of ligand molecule to be comprised in a pharmaceutical dosage unit according to the invention may be easily adapted by the one skilled in the art using routine known protocols or methods.

The ligand molecule and the pharmaceutical composition disclosed herein are administered by any suitable route, i.e. including, but not limited to, an oral, sublingual, buccal, subcutaneous, transdermal, topical, intraperitoneal, intramuscular, intravenous, subdermal, intrathecal and intranasal and rectal administration.

Advantageously, the method for treating a KIR3DL2 expressing malignant T-cells lymphoma in an individual in need thereof, as disclosed in the instant invention is capable of inducing at least about 10%, for example about 20%, for example about 30%, for example about 40%, for example about 50%, for example about 60%, for example about 70%, for example about 80%, for example about 90% of cell death.

### EXAMPLES

### EXAMPLE 1: Expression of KIR3DL2 in Cutaneous, Non-Cutaneous Peripheral Extra Nodal and Nodal T-cell Lymphomas

1) Material and Methods

1.1) Material and Methods

Tissue samples were retrieved from various collections, and diagnoses were done in all cases following the current classification (Swerdlow S H, Campo E, Harris N L, et al. In: press IARC, ed. WHO Classification of Tumours of Haematopoietic and Lymphoid tissues (ed 4th). Lyon; 2008). The international classification of diseases for oncology (ICDO) code is given in parenthesis for each lymphoma group disclosed in the following list.

a) Cutaneous T-cell Lymphomas

Frozen skin samples of patient with Sézary syndrome (9701/3) KI04028, KI10025 and KI18027 were collected from the national research project PHRC (programme hospitalier de recherche clinique) KIRs, with Dr Nicolas ORTONNE as principal investigator and coordinator. They were selected from patients with Sézary syndrome, in which strong KIR3DL2 mRNA transcript expression was demonstrated using quantitative RT-PCR both in the blood and in the skin, while no significant expression of KIR3DL1 was found. In addition, two other Sézary syndrome cases from the department of Pathology (routine practice) were studied, with both a skin and involved lymph node specimen.

Frozen skin samples from other cutaneous T-cell lymphomas were retrieved from the archival files of the department of Pathology of the hospital Henri Mondor (AP-HP, groupe hospitalier Henri Mondor Albert Chenevier). These samples included:
3 transformed mycosis fungoides (9700/3t);
4 primary cutaneous CD30+ T-cell lymphoproliferative disorders with 2 cutaneous anaplastic large cell lymphomas (9718/3) and 2 cases of lymphomatoid papulosis (9718/1);
4 sub-cutaneous panniculitis-like T-cell lymphomas (9708/3);
1 primary cutaneous nasal-type NK/T-cell lymphoma (9719/3);
2 primary cutaneous CD8+ aggressive epidermotropic cytotoxic T-cell lymphoma (9709/3).

b) Nodal and Non-Cutaneous extra Nodal Lymphomas

Frozen skin samples from nodal and non-cutaneous extra nodal lymphomas were collected from the research project PHRC TENOMIC, with Philippe GAULARD as principal investigator and coordinator. We selected cases with more than 70% of neoplastic cells in the routine histological control. These samples included:
4 angioimmunoblastic T-cell lymphomas (AITL, 9705/3): TENOMIC 060, 268, 424, 415;
3 anaplastic large cell lymphomas, ALK negative (9702/3): TENOMIC 105, 213, 238;
6 enteropathy-associated T-cell lymphomas (EATL, 9717/3): TENOMIC 046, 418, 441, 210, 319, 358, 413;
8 adult T-cell leukaemia/lymphoma (ATLL, 9827/3): TENOMIC 285, 257, 340, 361, 066, 256, 540, 560;
4 extra nodal NK/T cell lymphomas nasal-type (9719/3): TENOMIC 053, 246, 419, 579;
7 hepatosplenic gamma-delta T-cell lymphomas (HSTL, 9716/3): TENOMIC 014, 037, 181, 014, 037, 181, 183;
4 peripheral T-cell lymphomas, not otherwise specified (PTCL/NOS, 9702/3): TENOMIC 214 , 225, 232, 469.

In addition frozen sections of 5 anaplastic large cell lymphomas, ALK positive (9702/3) were obtained from Dr Laurence LAMANT, from the department of Pathology of the Institut Universitaire du Cancer de Toulouse—Oncopole: P9710730, P055401, P0016632, P00113872, P126370.

1.2) Immunohistochemistry

For immunostaining procedures, 3 μm-thick sections were applied on Superfrost plus slides (CML, Angers, France). In each case a section was stained with hematoxylin eosin and saffron (HES) to check the quality of the tissue samples (necrosis) and confirm the presence of the neoplastic infiltrate.

KIR3DL2 immunostaining was done manually in a humid chamber using a mouse IgG1 monoclonal antibody (clone 5.133, Miltenyi Biotec, Paris, France) ata 1:50 dilution with 1 hour incubation. This antibody reacts with both KIR3DL2 and IR3DL1. The staining was performed using the EnVision® amplification system conjugated to peroxydase (Dako SA, Glostrup, Denmark). The peroxydase reaction was revealed by aminoethylcarbazole and sections were counterstained in blue with hematoxylin.

Other immunostainings were done to check for the expression of the classical phenotypic markers in each case, including T-cell markers for all samples (CD3), CD30 for anaplastic large cell lymphomas and primary cutaneous CD30+ T-cell lymphoproliferative disorders, CD8 and granzyme B for sub-cutaneous panniculitis-like T-cell lymphomas and primary cutaneous CD8+aggressive epidermotropic cytotoxic T-cell lymphoma, CD56 and granzyme B for cutaneous and extra nodal NK/T cell lymphomas nasal-type, CXCL13 and PD1 for AITL, CD25 for ATLL, CD5 and TiAl for HSTL. These additional stainings were done either manually using a biotin/avidine system conjugated to peroxydase (Vectastain® ABC-P kit from Vector, Burlingame, USA) or using the Bond-Max automated device (Menarini, Leica). The peroxydase reaction was revealed by diaminobenzidine (Sigma-Aldrich, Saint Quentin Fallavier, France) and sections were counterstained in blue with hematoxylin.

Slides were analyzed using the Axioskop 2 microscope (Zeiss, Germany) and pictures were taken using the digital camera EOS 600D (Canon, France).

1.3) RT-PCR Studies

Quantitative PCR reactions for CD3 (delta chain), KIR3DL2 and KIR3DL1 were performed in a LightCycler 2.0 System (Roche Diagnostics, Meylan, France) using a SYBR Green PCR kit from Roche Diagnostics (Meylan, France). Melting curves and agarose gel electrophoresis established the purity of the amplified product. Normalization was achieved by quantification of the mRNA expression of the SF3A1 gene, encoding for the 120 kDa subunit of the splicing factor 3a, chosen as control housekeeping gene for its stable expression in lymphocytes, as previously described. PCR samples contained 4 mM MgCL₂, 0.4 μM of each primer, and amplification cycling conditions were as following: 94° C. for denaturation, 10 seconds at 60° C. for hybridization and 25 seconds at 72° C. for elongation for 40 cycles. The expression of transcripts was measured by the relative quantification of real time-PCR, as previously described. All PCR conditions were adjusted in order to obtain equivalent optimal amplification efficiency between the different assays. By using the obtained linear graphs, the differences in Cₜ values were determined for each sample and were expressed as relative percentage of mRNA present in the calibrator sample, according to the ΔΔCₜ method, after adjustment of PCR efficiency with the Light Cycler software 4.0 (Roche). Quantification was considered to be unreliable when the presence of non-specific products was detected on the control agarose gel.

Quantitative RT-PCR studies were done in the positively stained cutaneous T-cell lymphomas: one primary cutaneous CD8+ aggressive epidermotropic cytotoxic T-cell lymphoma and two sub-cutaneous panniculitis-like T-cell lymphomas.

Total RNA extraction was performed on frozen sections, transferred into Trizol, and immediately homogenized before chloroform/isopropanol precipitation. Total mRNA was then reverse transcribed by using the High Capacity cDNA Reverse Transcription with RNase inhibitor kit (Applied Biosystems), according to the manufacturer's instructions.

Primers used to quantify KIR3DL2 may be of SEQ ID NO: 1 and SEQ ID NO: 2:

SEQ ID NO: 1: forward
5′-CAACTTCTCCATCGGTCCCTTGATG-3′
SEQ ID NO: 2: reverse
5′-GTTTGACCACACGCAGGGCAG-3′.

Primers used to quantify KIR3DL1 may be of SEQ ID Nº3 and 4:

SEQ ID NO: 3: forward
5′-GGACATCGTGGTCACAGGTCC-3′
SEQ ID NO: 4: reverse
5′-GCCTGGAATGTTCTGTTGACCTTGC-3′.

Amplification was done using newly designed primers and SYBR® green, allowing the specific detection of KIR3DL1 and KIR3DL2 transcripts, as in a previous publication (Ortonne et al. CD158k/KIR3DL2 and NKp46 are frequently expressed in transformed mycosis fungoides. Exp Dermatol. 2012 Jun; 21(6):461-3) and the ABI 7900HT device (Applied Biosystems).

The SF3A1 housekeeping gene was used as calibrator. The levels of expression of the KIR3DL1 and KIR3DL2 receptors were finally expressed as a ratio to CD36 to avoid the potential bias due to differences in T-cell lymphocytic densities.

In two HSTL and one EATL and ATLL (PHRC TENOMIC), mRNA levels of K R3DL2 was studied by transcriptomic analysis (Affymetrix U133 Plus 2.0) after total mRNA extraction from frozen specimens and compared to all HSTL and AITL.

2) Results

2.1) Immunohistochemistry

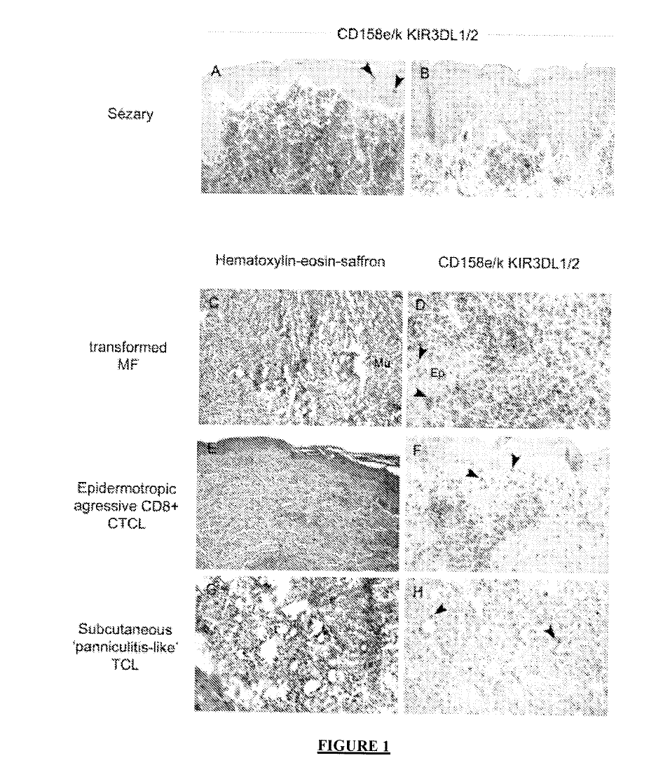

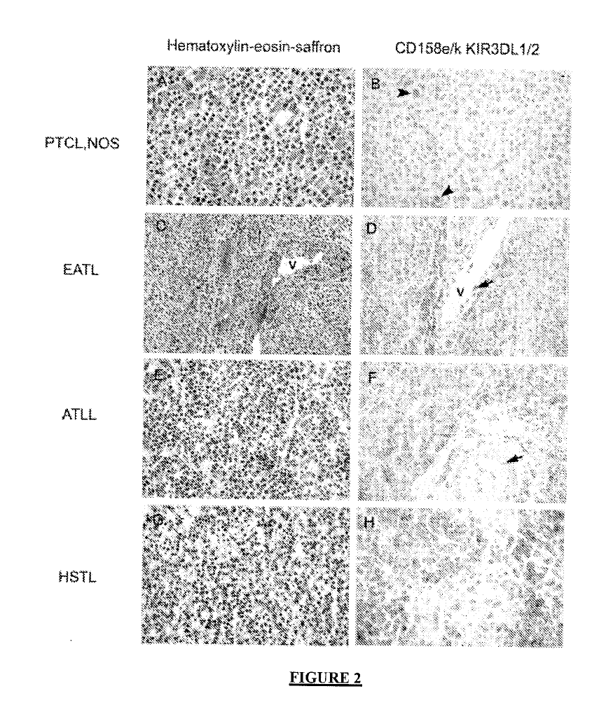

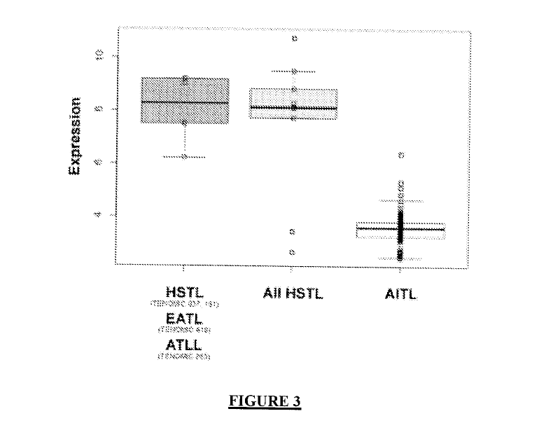

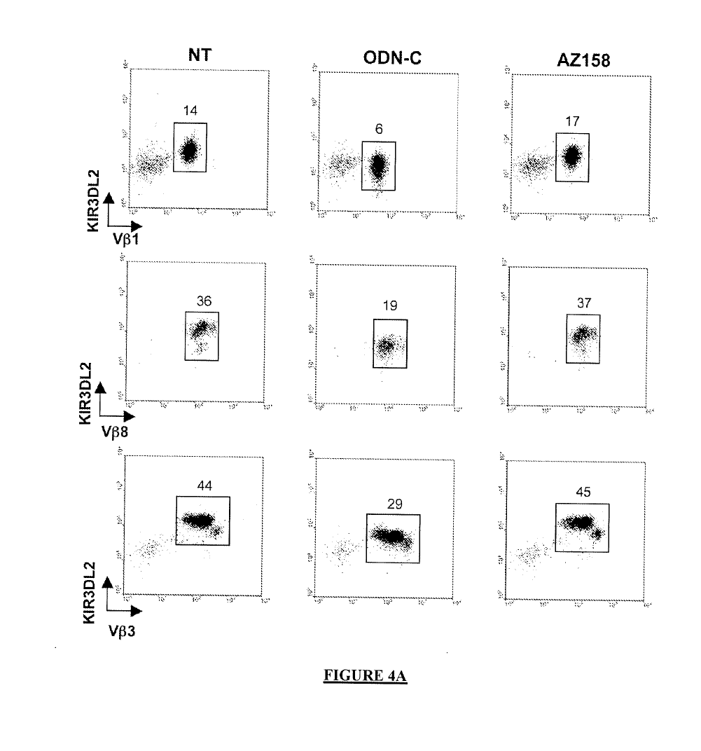

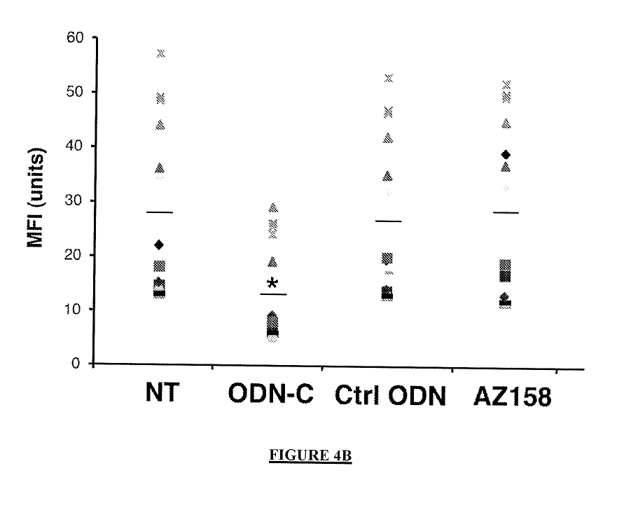

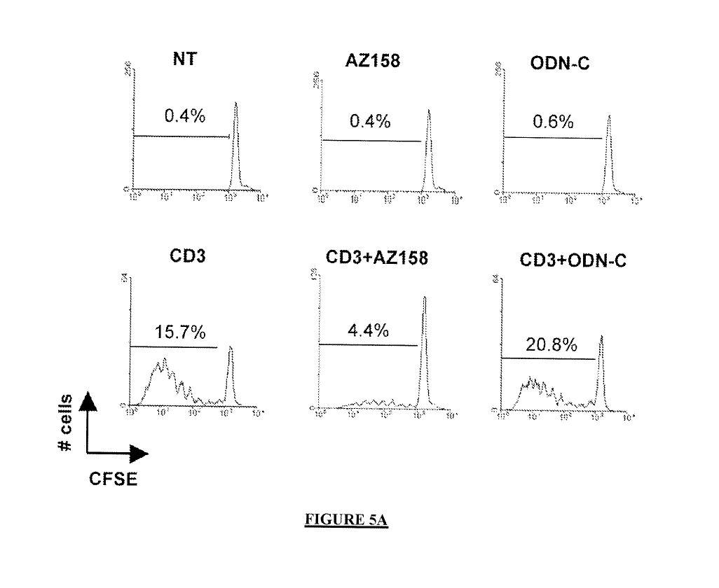

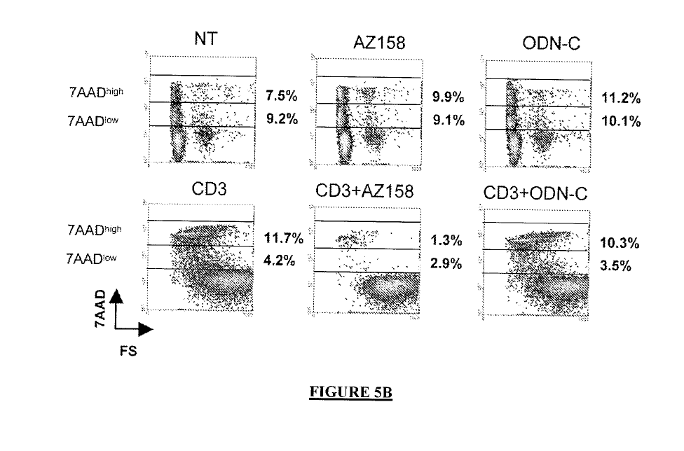

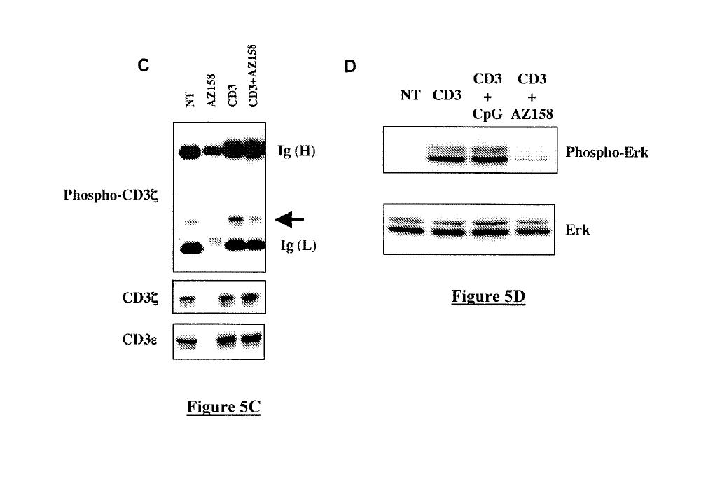

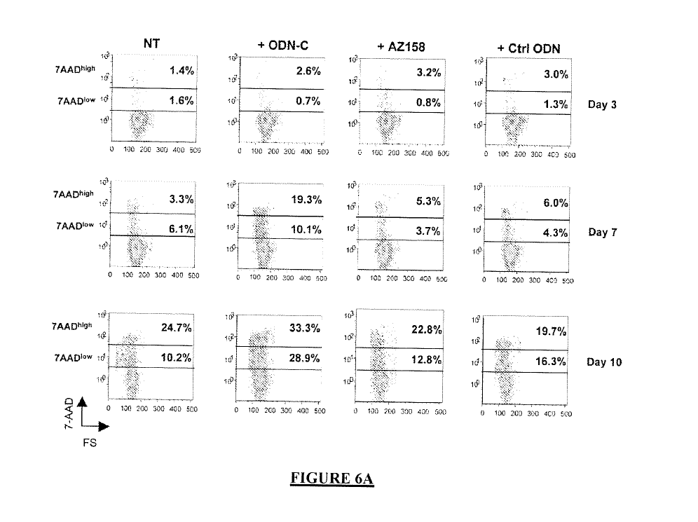

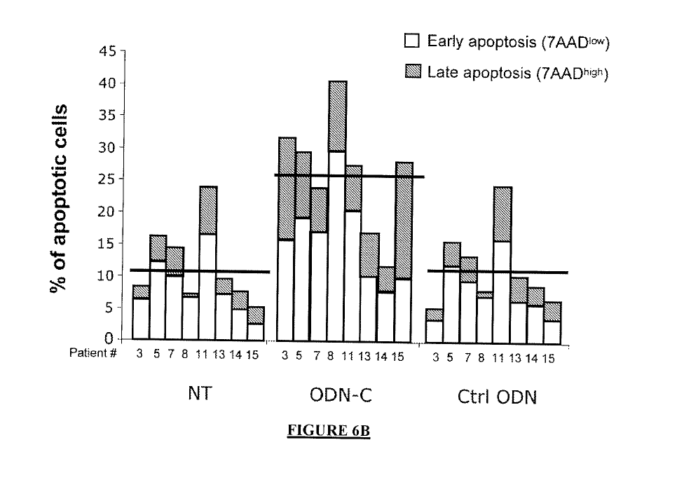

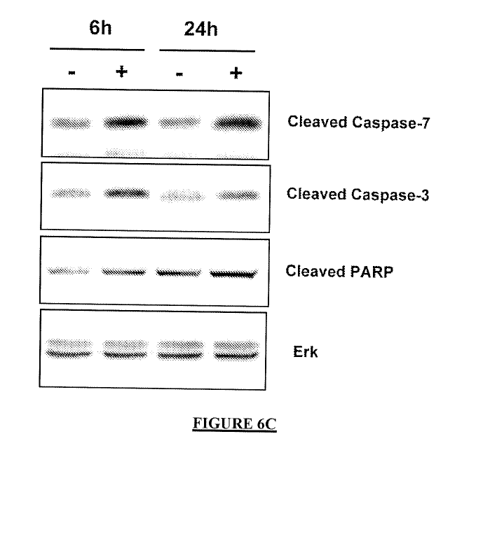

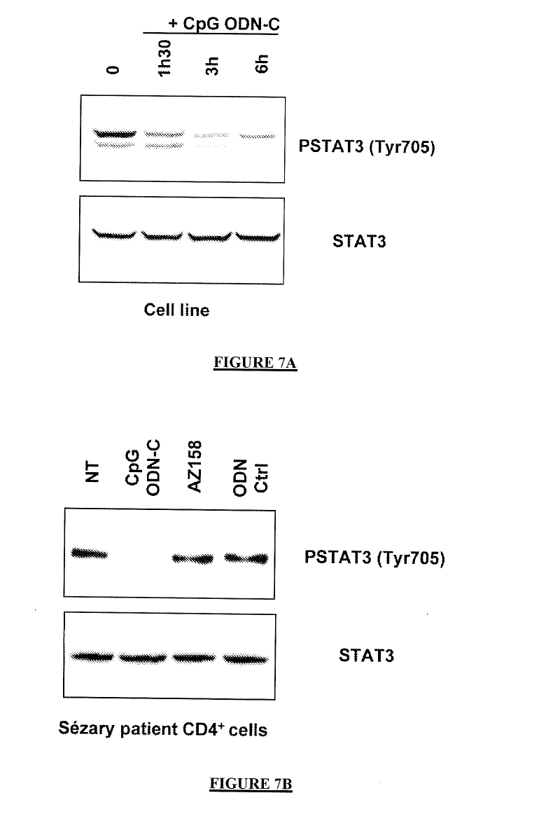

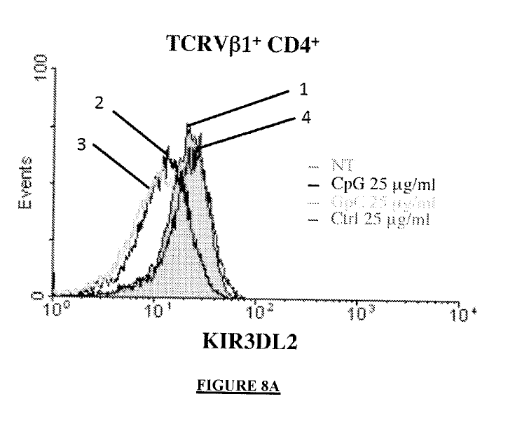

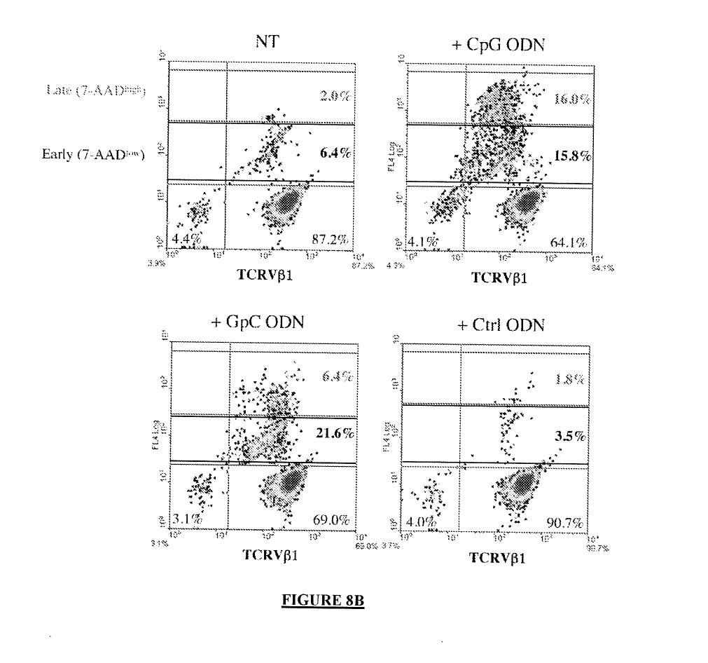

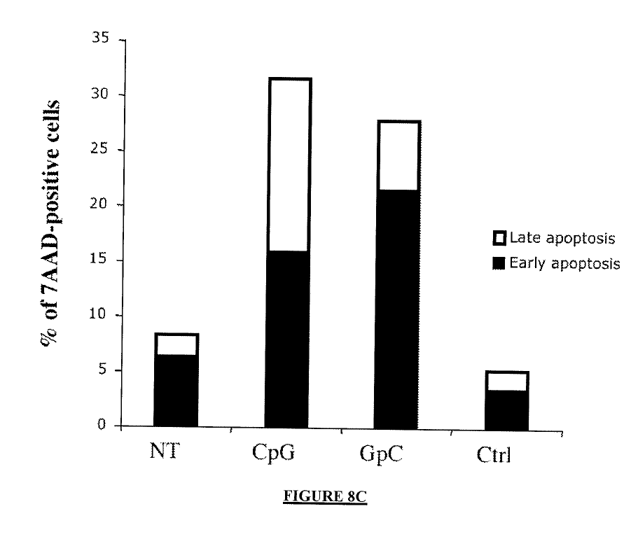

The results from FIGS. 1 and 2 are summarized in the Table 1 below.

TABLE 1
phenotypic study and proportion of cases displaying a positive ratio of
expression KIR3DL1/2 (cohort of 44 individuals).

| | | | | KIR3DL1/2+ |
|---|---|---|---|---|
| | | | | cases |
| | | | Number | using IHC² |
| Diagnosis | ICDO¹ | Phenotype | of cases | (%) |
| Sézary (skin) | 9701/3 | CD3+, CD8−, | 5 | 5 (100%) |
| | | PD1+ | | |
| Sézary (lymph node) | 9701/3 | CD3+, CD8−, | 2 | 2 (100%) |
| | | PD1+ | | |
| Transformed mycosis | 9700/3 | CD3+, CD4+ | 3 | 1 (33%) |
| fungoides | t | | | |
| Primary cutaneous | 9718/3, | CD3+, CD30+ | 4 | 0 |
| CD30+ T-cell | 9718/1 | | | |
| lymphoproliferative | | | | |
| disorders | | | | |
| Sub-cutaneous | 9708/3 | CD3+, CD8+, | 4 | 4 (100%)⁴ |
| panniculitis like T-cell | | GrB+³ | | |
| lymphomas | | | | |
| Primary cutaneous | 9709/3 | CD3+, CD8+, | 1 | 1 (100%) |
| CD8+ aggressive | | GrB+ | | |
| epidermotroptc | | | | |
| cytotoxic T-cell | | | | |
| lymphoma | | | | |
| Angioimmunoblastic | 9705/3 | CD3+, | 4 | 0 |
| T-cell lymphomas | | CXCL13+, | | |
| | | PD1+ | | |
| Anaplastic large cell | 9702/3 | CD3+, CD30+ | 3 | 0 |
| lymphomas, ALK | | | | |
| negative | | | | |
| Enteropathy-associated | 9717/3 | CD3+ | 3 | 1 (33%) |
| T-cell lymphomas | | | | |
| Adult T-cell | 9827/3 | CD3+, CD25+ | 4 | 1 (25%) |
| leukaemia/lymphoma | | | | |
| Extranodal NK/T cell | 9719/3 | CD3c+, CD56+, | 4 | 0 |
| lymphomas nasal-type⁵ | | GrB+, EBV+⁶ | | |
| Hepatosplenic gamma- | 9716/3 | CD3+, CD5−, | 3 | 3 (100%) |
| delta T-cell lymphomas | | TiAl+ | | |
| Peripheral T-cell | 9702/3 | CD3+ | 4 | 0 |
| lymphomas, not | | | | |
| otherwise specified | | | | |

¹ICDO stands for International Classification of Diseases for Oncology;
²IHC stands for ImmunoHistoChemistry;
³GrB stands for granzyme B;
⁴Only scattered positive cells were identified, which may be neoplastic and/or reactive;
⁵One case was a primary cutaneous form, and the three others were from the nasal cavity;
⁶EBER transcripts.

In all cases, the standard staining with HES showed no or only little tissue necrosis and presence of the T-cell neoplastic infiltrate. Expression of classical phenotypic markers was demonstrated in all samples (data not shown).

KIR3DL1/2 staining in all the Sézary syndrome skin samples showed as expected a membrane staining in the neoplastic infiltrates, with no background staining (FIGS. 1A and 1B). No staining was evidence in irrelevant structures, including the epidermis, the cutaneous adenexae, normal dermal cells and hypodermis. A strong and diffuse staining was also evidenced in the lymph nodes from two cases.

In the cutaneous T-cell lymphomas group, a significant diffuse and strong staining was evidenced in a transformed mycosis fungoides (n=⅓, FIGS. 1C and 1D) and in the primary cutaneous CD8+ aggressive epidermotropic cytotoxic T-cell lymphoma (FIGS. 1E and 1F). In all sub-cutaneous panniculitis-like T-cell lymphomas, scattered KIR3DL1/2+ cells were present but the staining was not positive in the majority of the neoplastic cells that are mostly localized around adipocytes.

In the peripheral T-cell lymphoma group, most cases were not stained with only scattered KIR3DL1/2+ cells, as shown for a PTCL/NOS sample in FIGS. 2B. One case of EATL (n=⅓, FIGS. 2C and 2D) and one case of ATLL (n=¼, FIGS. 2E and 2F) showed strong and diffuse KIR3DL1/2 expression, while all samples of HSTL studied showed a diffuse and strong KIR3DL1/2 expression (FIGS. 2G and 2H).

TABLE 2
phenotypic study and proportion of cases displaying a positive ratio of
expression KIR3DL1/2 (as in Table 1, but with results obtained from an
additional cohort of 16 individuals).

| | | | | KIR3DL1/2+ |
|---|---|---|---|---|
| | | | | cases |
| | | | Number | using IHC² |
| Diagnosis | ICDO | Phenotype | of cases | (%) |
| Sézary (skin) | 9701/3 | CD3+, CD8−, | 5 | 5 (100%) |
| | | PD1+ | | |
| Sézary (lymph node) | 9701/3 | CD3+, CD8−, | 2 | 2 (100%) |
| | | PD1+ | | |
| Transformed mycosis | 9700/3 | CD3+, CD4+ | 3 | 1 (33%) |
| fungoides | t | | | |
| Primary cutaneous | 9718/3, | CD3+, CD30+ | 4 | 0 |
| CD30+ T-cell | 9718/1 | | | |
| lymphoproliferative | | | | |
| disorders | | | | |
| Sub-cutaneous | 9708/3 | CD3+, CD8+, | 4 | 4 (100%)⁴ |
| panniculitis-like T-cell | | GrB+³ | | |
| lymphomas | | | | |
| Primary cutaneous | 9709/3 | CD3+, CD8+, | 2 | 2 (100%) |
| CD8+ aggressive | | GrB+ | | |
| epidermotropic | | | | |
| cytotoxic T-cell | | | | |
| lymphoma | | | | |
| Angioimmunoblastic | 9705/3 | CD3+, | 4 | 0 |
| T-cell lymphomas | | CXCL13+, | | |
| | | PD1+ | | |
| Anaplastic large cell | 9702/3 | CD3+, CD30+ | 3 | 0 |
| lymphomas, ALK | | | | |
| negative | | | | |
| Anaplastic large cell | 9702/3 | CD3+, CD30+ | 5 | 0 (0%) |
| lymphomas, ALK | | | | |
| positive | | | | |
| Enteropathy-associated | 9717/3 | CD3+ | 6 | 3 (50%) |
| T-cell lymphomas | | | | |
| Adult T-cell | 9827/3 | CD3+, CD25+ | 8 | 3 (37.5%) |
| leukaemia/lymphoma | | | | |
| Extranodal NK/T cell | 9719/3 | CD3c+, CD56+, | 4 | 0 |
| lymphomas nasal-type⁵ | | GrB+, EBV+⁶ | | |
| Hepatosplenic gamma- | 9716/3 | CD3+, CD5−, | 7 | 6 (86%) |
| delta T-cell lymphomas | | TiAl+ | | |
| Peripheral T-cell | 9702/3 | CD3+ | 4 | 0 |
| lymphomas, not | | | | |
| otherwise specified | | | | |

Results obtained by a cohort of 16 additional individuals confirmed that KIR3DL2 is a relevant biomarker for:
two cutaneous PTCLs, namely sub-cutaneous panniculitis-like T-cell lymphoma and primary cutaneous CD8+ aggressive epidermotropic cytotoxic T-cell lymphoma, and
two non-cutaneous PTCLs, namely enteropathy-associated T-cell lymphoma and hepatosplenic gamma-delta T-cell lymphoma.

KIR3DL2 may thus be a biomarker for diagnosing a sub-population of patients having a lymphoma such as a transformed mycosis fungoides, an enteropathy-associated T-cell lymphoma and an adult T-cell leukaemia/lymphoma. It could also be suggested that KIR3DL2 might be a good candidate to assess the prognosis of these particular cutaneous and non-cutaneous nodal and extra nodal lymphomas.

2.2) RT-PCR Studies

Quantitative RT-PCR studies showed significant expression of KIR3DL2 with markedly less KIR3DL1 in all cutaneous T-cell lymphomas studied (Table 3).

TABLE 3
RT-PCR studies for KIR3DL1 and KIR3DL2 in positively stained
cutaneous T-cell lymphomas.

| | | KIR3DL2/ | KIR3DL1/ | KIR3DL2/ |
|---|---|---|---|---|
| | | CD3δ | CD3δ | KIR3DL1 |
| Diagnosis | Case | ΔΔCₜ values | ΔΔCₜ values | ratios |
| Sézary (skin) | 1 | 5.83 | 0.03 | 194.33 |
| | 2 | 31.6 | 0 | ∞ |
| | 3 | 6.4 | 0.43 | 14.88 |
| Transformed | 1 | 15.16 | 0.23 | 65.91 |
| mycosis | | | | |
| fungoides | | | | |
| Sub-cutaneous | 1 | 0.95 | 0.28 | 3.39 |
| panniculitis-like | 2 | 9.83 | 1.01 | 9.73 |
| T-cell lymphomas | | | | |
| Primary cutaneous | 1 | 18.22 | 0.14 | 130.14 |
| CD8+ aggressive | | | | |
| epidermotropic | | | | |
| cytotoxic T-cell | | | | |
| lymphoma | | | | |

RT-PCR experiments were conducted with specific primers, as previously done (Ortonne et al. CD158K/KIR3DL2 Transcript detection in lesional skin of patients with erythroderma is a tool for the diagnosis of Sezary syndrome. J Invest Dermatol 2007; 128: 465-72). The expression of each receptor was calibrated with the SF3A1 housekeeping gene, and the data shown in column 3 and 4 represent the ratios of the ΔΔCₜ values between each receptor and the CD36 chain.

The KIR3DL2 ΔΔCₜ values (×10) for the Sézary syndrome samples were 5.83, 31.6 and 6.4, while for KIR3DL1, the values were 0.03, 0 and 0.43, respectively. Similarly, in the positively stained transformed mycosis fungoides, the ΔΔCₜ values were 15.16 for KIR3DL2 and 0.23 for KIR3DL1. In the primary cutaneous CD8+ aggressive epidermotropic cytotoxic T-cell lymphoma, KIR3DL2 transcripts also appeared to be expressed at a much higher rate than KIR3DL1, with a KIR3D2/KIR3DL1 ratio of 130.14.

In the two subcutanous panniculitis-like lymphomas, KIR3DL2 transcripts were also more expressed than but the difference with KIR3DL1 was less marked. It can be therefore conclude that these cases rather expressed KIR3DL2 than KIR3DL1 and that the anti-KIR3DL1/2 clone 5.133 stained KIR3DL2 at the surface of the neoplastic cells. In 2 HSTL samples, and in the EATL and ATLL samples showing a positive staining, transcriptomic analyzes also identified significant KIR3DL2 mRNA expression, at much higher levels than in other peripheral T-cell lymphomas, as shown in FIG. 3.

It is to be noted that the present results seem contradictory with the results obtained by Iqbal et al. (Molecular signature to improve diagnosis in peripheral T-cell lymphoma and prognostication in angioimmunoblastic T-cell lymphoma; Blood, 2010, 115(5):1026-36) reported KIR3DL2 as a putative biomarker for a subset of PTCL “not otherwise specified” (PTCL/NOS). However, one may observe that the PTCL/NOS encompass a variety of lymphomas of distinct nature, as they could not be assigned to one specific defined subcategory.

3) Conclusion

A subset of cutaneous T-cell lymphomas (sub-cutaneous panniculitis-like T-cell lymphomas, and primary cutaneous CD8+ aggressive epidermotropic cytotoxic T-cell lymphoma) and extra-cutaneous peripheral T-cell lymphomas (some hepatosplenic T-cell lymphomas and enteropathy-associated T-cell lymphomas) expressed KIR3DL2 and thus appear to be good candidates for a targeted therapy.

With the exception of subcutaneous ‘panniculitis-like’ T-cell lymphomas, the vast majority of neoplastic cells were stained with the anti-KIR3DL1/2 monoclonal antibody. In all the cases with available RT-PCR results KIR3DL2 transcripts were detected, at much higher levels than KIR3DL1. The identification of KIR3DL2 expression in tissue samples using the anti-human KIR3DL1/2 mouse IgGI monoclonal antibody (clone 5.133, Miltenyi Biotec, Paris, France) was reliable for the identification of KIR3DL2+ lymphomas.

### EXAMPLE 2: Monoclonal Antibody (MAb) AZ158 or CpG ODN Binding to KIR3DL2 Induces Distinct Cellular Death Pathways in Sézary Syndrome Malignant T Cells

1) Materials and Methods

1.1) Patients and Cells

Sézary syndrome diagnosis was established on recognized international clinical, histological and biological criteria. Blood from 15 Sézary syndrome patients with more than 90% of CD3⁺CD4⁺KIR3DL2⁺ cells was collected for the present study, which was approved by the institutional ethics committee (Saint Louis Hospital, Paris).

Peripheral blood mononuclear cells (PBMCs) were isolated from heparinized venous blood by density gradient centrifugation over lymphocytes separating medium (LSM; PAA Laboratories, Les Mureaux, France).

CD4⁺ T cells were purified by MACS using the CD4⁺ T cell isolation kit according to the manufacturers protocol (Miltenyi Biotech). The Sézary cell line used in this study was established and amplified as described previously, and maintained a stable phenotype. Cells were cultured in RPMI 1640 medium, supplemented with 2 mM L-glutamine, 1% penicillin-streptomycin (Invitrogen) and 10% human serum (Jacques Boy Biotechnologies Institute).

1.2) CpG ODN. GpC ODN or Antibodies Cell Treatment

For CpG ODN and GpC ODN treatments, cells were cultured for the indicated time (1, 4 or 7 days) in 24-well plates at a concentration of 2×10⁶/ml. The following CpG ODNs and or GpC ODN were used at a final concentration of 10 μg/ml: CpG class-A (ODN 2336), CpG class-B (ODN 2006), CpG class-C (ODN 2395), GpC (ODN 2395 control) and control ODN (ODN TTAGGG) (all from Invivogen).

TABLE 4
Sequences of the CpG ODNs and GpC ODN used:

| SEQ ID NO | CpG ODN/GpC ODN | 5′-3′ sequence¹ |
|---|---|---|
| SEQ ID NO: 5 | CpG class-A (ODN 2336) | gggGACGACGTCGTGgggggg |
| SEQ ID NO: 6 | CpG class-B (ODN 2006) | tcgtcgttttgtcgttttgtcgtt |
| SEQ ID NO: 7 | CpG class-C (ODN 2395) | tcgtcgttttcggcgcgcgccg |
| SEQ ID NO: 8 | GpC (ODN 2395 control) | tgctgcttttggggggcccccc |

¹Bases in capital letters are phosphodiester, those in lower case are phosphorothioate.

Incubation was processed for up to 12 days.

For short-time stimulation, cells were left untreated or treated with anti-CD3 (CD3×3, IgGI; locally produced), anti-KIR3DL2 (AZ158, IgG2a; kindly provided by Innate Pharma, Marseille, France) or CpG ODN-C alone or in combination, followed by cross-linking with goat anti-mouse IgG Ab 5 min at 37° C. (Beckman Coulter).

Proliferation and apoptosis assays were performed on cells cultured for 4 (FIG. 5) or 7 days (FIGS. 6 and 8) at 37° C. in 96-well plates pre-coated with the indicated antibodies CpG ODN and/or GpC ODN. After incubation, cells were processed for flow cytometry or biochemical analysis, as described below.

1.3) Flow Cytometry

Sézary cell staining was performed according to a standard procedure using the anti- KIR3DL2 mAb Q66 (IgM; kindly provided by Dr A. Moretta, Genova, Italy) plus goat anti-mouse IgM-FITC antibodies, anti-CD3-PC7, -TCRV-PE and -CD4-PC5 mAbs (Beckman Coulter, Marseille, France). For proliferation assays, cells were pre-loaded with 0.5 μM of CFSE (Invitrogen) prior to activation while detection of apoptotic cells was performed using PE-conjugated 7AAD (BD Biosciences), according to the suppliers' protocols. Cells were analysed on an FC500 cytometer (Beckman Coulter).

1.4) Immunofluorescence

Cells were either left untreated or incubated in the presence of CpG ODN-C, FITC-labelled CpG ODN-C or control ODN for 24 h at 37° C. Cells were subjected to KIR3DL2 immunolabelling with Q66 mAb and FITC-coupled goat anti-mouse IgM Abs, washed and immobilized on poly-L-lysine coated coverslips. After a methanol fixation step at −20° C., cells were mounted in polyvinyl alcohol mounting medium with DABCO (Fluka). For KIR3DL2 intracellular staining, cells were adhered to poly-L lysine coverslips, fixed in methanol and permeabilized with PBS/0.1% Tween 20 prior to KIR3DL2 labelling. After washes, coverslips were mounted and analysed on a Leica DMRB microscope.

1.5) Immunoprecipitation and Western Blotting

Activated cells were subjected to lysis and post-nuclear supernatants prepared and processed as described elsewhere. For Western blotting, samples were separated by SDS-PAGE and transferred onto a nitrocellulose membrane. Immunoprecipitates were probed with anti-phospho-CD3 mAb (Santa Cruz Biotechnology, Santa Cruz, Calif.) and anti-CD3 mAb (Cell Signaling). Post-nuclear lysates analyses were performed using antibodies specific for the following molecules: cleaved-caspase 3, -caspase 7 and -PARP, phospho-STAT3, STAT3, Erk1/2 (all from Cell Signaling Technology) and phospho-Erk1/2 (Sigma-Aldrich). When performing detection in series, a stripping procedure between each revelation step was done. After incubation with the appropriate horseradish peroxidase-conjugated secondary antibodies (Jackson Immunoresearch), the detection was performed using an ECL system (Perbio Science, Brébières, France) and an ImageQuant LAS 400 system (GE Healthcare).

2) Results

2.1) KIR3DL2 Internalization is Induced Upon its Engagement with CpG ODN, but not with Anti-KIR3DL2 mAb AZ158

We previously identified KIR3DL2 as a reliable cell surface marker of the tumoral CD4⁺ T lymphocytes of patients with Sezary syndrome. To establish if KIR3DL2 exerts any function in Sézary CD4⁺ T cells, engagement of the receptor was achieved by using either the anti-KIR3DL2 monoclonal antibody (mAb) AZ158 or its newly identified ligand CpG ODN. Note that both AZ158 and CpG ODN-C sites of interaction were mapped within the DO extracellular domain of the receptor.

Because CpG ODNs were shown to promote KIR3DL2 cell surface down-modulation on NK cells (Sivori et al. A novel KIR-associated function: evidence that CpG DNA uptake and shuttling to early endosomes is mediated by KIR3DL2; Blood, 2010, 116(10):1637-1647), we first tested whether such observation also applied to malignant Sézary cells. We observed that incubation of Sezary patients PBMCs with type A, B or C CpG ODNs, but not with a control ODN, led to a substantial down-modulation of KIR3DL2, corresponding to a 50% reduction of the receptor mean fluorescence intensity, at the surface of Sézary patients CD4⁺ T cells (Table 5).

TABLE 5
Down-modulation of KIR3DL2 by CpG ODN on Sézary patients
malignant T cell clone.

| | MFI KIR3DL2 (units) | | | | |
|---|---|---|---|---|---|
| Patient | NT¹ | CpG ODN- | CpG ODN-B | CpG ODN-C | Ctrl² CpG |
| P7 | 34 | 25 | 18 | 17 | 35 |
| P13 | 143 | 79 | 81 | 60 | 135 |
| P14 | 129 | 90 | 48 | 49 | 132 |
| P15 | 68 | 36 | 38 | 55 | 70 |
| Mean MFI | 94 | 58 | 46 | 45 | 93 |

¹not treated;
²control.

Because CpG ODN-C induced efficient cell surface modulation of KIR3DL2 and combined the immune effects of class-A and -B ODN (CpG ODN-A et CpG ODN-B) on immune cells it was preferentially used for the following experiments. The expression level of KIR3DL2 by Sézary patients tumoral T cell clone was next monitored in parallel on ODN-C or AZ158 mAb treated cells. The resulting data clearly demonstrated that while ODN-C promoted KIR3DL2 down-modulation, ligation of KIR3DL2 with AZ158 mAb did not affect the level of receptor detected on malignant T cells (identified by means of their clonal TCRVI3 rearrangement) (FIG. 4A). Identical results were obtained in 12 different Sézary patients who were analysed (FIG. 4B).

Finally, fluorescence microscopy analysis on an established Sézary cell line showed that KIR3DL2, which is evenly distributed at the plasma membrane in resting cells or after contact with a control ODN, is partially internalized following CpG ODN-C cell treatment. Similarly, when using a FITC-conjugated CpG ODN-C, this latter was detected both at the plasma membrane and at the intracellular level after cell incubation. Altogether, these data established that binding of CpG ODN-C, but not of AZ158 mAb, to KIR3DL2 promotes the receptor internalization in Sézary cells.

2.2) AZ158 mAb, but not CpG ODN-C, Promotes KIR3DL2 Co-Receptor Inhibitory Function To further investigate the consequences of KIR3DL2 triggering on Sézary cells, we first evaluated its potential function on the processes of CD3-induced malignant T cell proliferation and apoptosis. To this aim, PBMCs from Sézary patients were activated for 4 days with an anti-CD3 mAb alone or in association with AZ158 mAb or CpG ODN-C. The malignant T cell clone proliferation and apoptosis status was further assessed by immunolabelling. Representative results obtained on PBMCs from patient 1 are shown in FIG. 5A and B. We observed that the CD3-dependent proliferation of the malignant T cell clone (identified as TCRV8⁺CD4⁺ T cells) was strongly inhibited upon binding of AZ158 mAb to KIR3DL2 but not with CpG ODN-C (FIG. 5A). Consequently, the CD3-induced tumoral cell death was also found impaired in the presence of AZ158 mAb while CpG ODN-C had no effect (FIG. 5B). In agreement with these observations, a down-modulation of the CD3 chain phosphorylation and Erk1/2 activation, that is induced upon CD3 targeting, was observed when AZ158 mAb was bound to KIR3DL2 (FIG. 5, C and D). In contrast, no notable modification of the CD3-mediated Erkl/2 activation was observed in the presence of CpG ODN-C. Thus, it seems that binding of CpG ODN-C to KIR3DL2 did not interfere with the CD3-dependent activation process while binding of AZ158 mAb to KIR3DL2 revealed its co-receptor inhibitory function.

2.3) Long-term CpG ODN-C/KIR3DL2 Interaction Leads to Sézary Cells Apoptosis

We initially did not observe any impact of CpG ODN-C on the tumoral cell viability when performing a 4 days-incubation assay (see FIG. 5B). Incubation times were therefore extended to up to 12-days. Representative results obtained on PBMCs from patient 15 at day 7 are shown in FIG. 6A.

Remarkably, at day 7 while the tumoral cell viability was not affected by the length of the treatment (non-treated/NT panel) or by the presence of AZ158 mAb or control ODN (all conditions resulting in the detection of 5 to 6% of spontaneous apoptosis), addition of CpG ODN-C led to a significant increase in the malignant cell mortality, with the detection of 29% of 7AAD-positive cells within the tumoral population.

In contrast, similar experiments performed on PBMCs isolated from healthy donors revealed that although normal CD4⁺ T-cells appeared to be more sensitive to the experimental time length, with 15% of this population undergoing apoptosis during the time course, cell death was not amplified by the presence of CpG ODN-C or AZ158 mAb.

Results obtained with patient 15 (FIG. 6A) were confirmed with 7 additional patients (FIG. 6B), with maximal CpG ODN-C induced apoptosis detected after 7 to 12-days of treatment according to the patient.

Finally, the induction of a caspase-dependent apoptotic pathway following incubation with CpG ODN-C was confirmed by immunoblotting. As shown in FIG. 6C, the levels of cleaved forms of caspase-7 and -3, and of their substrate PARP, were found increased upon incubation of a Sézary cell line with CpG ODN-C, with maximum levels being reached after 6 h of treatment. Altogether, these results established that a specific apoptosis of Sézary malignant cells could be induced through treatment with CpG ODN-C.

2.4) CpG ODN-C Treatment of Sézary Cells Results in Phospho-STAT3 Dephosphorylation Previous studies have established that STAT3 can be constitutively phosphorylated in Sézary cells and that treatments promoting its dephosphorylation led to malignant cell death. We therefore investigated the phosphorylation status of STAT3 in CpG ODN-C treated cells. Kinetic experiments performed on a Sézary cell line showed that a significant time-dependent STAT3 dephosphorylation occurred upon CpG ODN treatment that is not correlated to the degradation of the protein (FIG. 7A). This dephosphorylation process was further confirmed on sorted CD4⁺ T-cells of patients whose all CD4⁺ T-lymphocytes were KIR3DL2 (FIG. 7B). In these cells, a complete dephosphorylation of STAT3 was visualized after 24 h of ODN-C incubation while phospho-STAT3 level was not modified in the presence of AZ158 mAb or control ODN. It therefore appeared that the CpG ODN-C-induced apoptosis of Sézary cells might be correlated to STAT3 dephosphorylation.

2.5) GpC ODN Treatment

GpC ODN differs from CpG ODN by the presence of GpC dinucleotides instead of CpGs, and is usually used as a negative control for CpG ODN.

Unexpectedly, a treatment of Sézary cells cultured for 7 days in the presence of GpC ODN was hereby shown to be as active on Sézary cells apoptosis as a treatment of Sézary cells cultured with CpG ODN-C (see FIG. 8A through 8C).

Both CpG ODN-C (FIG. 8A, curve 2) and GpC (FIG. 8A, curve 3) treatment of Sézary cells results in a decrease of KIR3DL2 expression as compared to Sézary cells either left untreated (FIG. 8A, curve 1) or treated with the control ODN (FIG. 8A, curve 4).

However, the effect of GpC ODN on Sézary cells seems different as compared to CpG ODN.

Indeed, early apoptosis represents 21% and late apoptosis 6% of the apoptosis events in Sézary cells treated with GpC ODN, whereas Sézary cells treated with CpG ODN-C display as many early apoptosis events as late apoptosis events (16%) (FIGS. 8B and 8C).

3) Conclusion

While binding to the same extracellular domain of KIR3DL2, namely the DO domain, AZ158 mAb and CpG ODN effects on Sézary cells are clearly distinct. We showed that the co-engagement of CD3 and KIR3DL2 through CpG ODN did not lead to the delivery of KIR3DL2-dependent negative signals. Moreover, we observed that unlike AZ158 mAb, CpG ODN promote the internalization of the receptor. For all patients tested, we experienced an averaged 50% reduction of KIR3DL2 MFI following CpG ODN treatment, this threshold not being over-passed when using higher concentrations of CpG ODN (up to 25 μg/ml) or longer exposure time (up to 12 days) (data not shown). In NK cells, KIR3DL2 internalization leads to the co-localization of the CpG ODN-linked receptors with TLR9 in the endosomal compartment 14. It has therefore been suggested that in these cells, KIR3DL2 may act as a carrier protein that brings CpG ODN to their receptor TLR9, resulting in NK cell activation. Despite the detection of TLR9 transcripts in Sézary cell lines and Sézary patient tumoral cells, we did not detect any TLR9 expression in these cells (data not shown). In addition, it has been recently reported that CpG and non-CpG ODN can co-stimulate mouse and human CD4+ T cells through a TLR9- and MyD88-independent mechanism 28. Together with our observations, these data suggested the possibility of a KIR3DL2/ODN CpG-mediated effect with no TLR9 involvement in Sézary cells. This also points towards a distinct role for KIR3DL2 as CpG ODN receptor in NK and Sézary cells (activating receptor vs apoptosis-mediating receptor, respectively) depending on TLR9 expression.

Unexpectedly, while maximal but only partial internalization of KIR3DL2 (corresponding to a loss of 50% of the receptor at the cell surface) and complete dephosphorylation of STAT3 are observed 24 h after exposure of Sézary patients tumoral cells to CpG ODN-C, detection of the tumoral T cell clone apoptosis requires 7 to 12 days of treatment. In contrast, experiments performed on proliferating Sézary cell lines showed a complete internalization of the receptor 2 to 4 h after addition of the CpG ODN (data not shown) with STAT3 dephosphorylation and cell apoptosis detected 6 to 8 h later (FIG. 4A and S1). It remains to determine if these time course discrepancies are the consequence of peripheral blood tumoral cells low metabolic turnover, as compared to an established and proliferating Sézary cell line, and the need for days to shutdown phospho-STAT3-dependent anti-apoptotic pathways. Nevertheless, the results show that in Sézary cells, KIR3DL2/CpG ODN association and internalization led to phospho-STAT3 dephosphorylation and tumoral cell death. Previous studies demonstrated that treatment of Sézary cells with the Janus kinase (JAK) inhibitor tyrphostin AG490, as well as with Cucurbitacin I or Curcumin, efficiently promotes phospho-STAT3 dephosphorylation and induces Sézary cell apoptosis. It has been recently established that constitutive activation of STATS in Sézary cells is not due to a loss of SHP-1, but is mediated by a constitutive aberrant activation of JAK family members. Efforts are currently made to determine how CpG ODN binding to KIR3DL2 in Sézary cells may influence JAK activity.

1-22. (canceled)

23. A method for diagnosing and treating a KIR3DL2 expressing malignant T-cells lymphoma being selected from the group comprising Sézary syndrome, transformed mycosis fungoides, sub-cutaneous panniculitis-like T-cell lymphoma, primary cutaneous CD8⁺ aggressive epidermotropic cytotoxic T-cell lymphoma, enteropathy-associated T-cell lymphoma and hepatosplenic gamma-delta T-cell lymphoma, in an individual in need thereof, said method comprising the step of:
a) providing a biological sample from the said individual,
b) measuring in the said biological sample the level of cellular protein expression of KIR3DL2, by the mean of a ligand molecule that specifically binds to KIR3DL2,
c) comparing the said level with a reference value,
d) determining said individual has said KIR3DL2 expressing malignant T-cells lymphoma, when the measured level of cellular protein expression of KIR3DL2 is higher than said reference value, and
e) treating said individual determined in step d) to have a KIR3DL2 expressing malignant T-cells lymphoma, with a ligand molecule that specifically binds to KIR3DL2.

24. The method according to claim 23, wherein the level of cellular protein expression of KIR3DL2 is quantified by measuring the level of protein surface expression.

25. The method according to claim 23, wherein said ligand molecule is selected from the group comprising an antibody, a fragment of an antibody and an oligodeoxynucleotide.

26. The method according to claim 25, wherein the oligodeoxynucleotide is selected from the group comprising CpG ODN-A of sequence SEQ ID NO: 5, CpG ODN-B of sequence SEQ ID NO: 6, CpG ODN-C of sequence SEQ ID NO: 7 and GpC ODN of sequence SEQ ID NO: 8.

27. A method for treating a KIR3DL2 expressing malignant T-cells lymphoma being selected from the group comprising Sézary syndrome, transformed mycosis fungoides, sub-cutaneous panniculitis-like T-cell lymphoma, primary cutaneous CD8⁺ aggressive epidermotropic cytotoxic T-cell lymphoma, enteropathy-associated T-cell lymphoma, adult T-cell leukaemia/lymphoma and hepatosplenic gamma-delta T-cell lymphoma, in an individual having a KIR3DL2 expressing malignant T-cells lymphoma comprising a step of administering a ligand molecule that specifically binds to KIR3DL2.

28. The method according to claim 27, wherein said ligand molecule is selected from the group comprising an antibody, a fragment of an antibody and an oligodeoxynucleotide.

29. The method according to claim 28, wherein the oligodeoxynucleotide is selected from the group comprising CpG ODN-A of sequence SEQ ID NO: 5, CpG ODN-B of sequence SEQ ID NO: 6, CpG ODN-C of sequence SEQ ID NO: 7 and GpC ODN of sequence SEQ ID NO: 8.
